(19) United States
(12) Patent Application Publication
(10) Pub. No.: US 2026/0108735 A1
(43) Pub. Date: Apr. 23, 2026

(54) SYSTEMS AND METHODS FOR PSEUDOMONOPHASIC BRAIN STIMULATION

(71) Applicant: Blackrock Microsystems, Inc., Salt Lake City, UT (US)

(72) Inventors: Sherman Perry WIEBE, Brussels (BE); Bryan HOWELL, Cary, NC (US)

(21) Appl. No.: 19/364,979

(22) Filed: Oct. 21, 2025

Related U.S. Application Data
(60) Provisional application No. 63/711,028, filed on Oct. 23, 2024.

(51) Int. Cl. A61N 1/36; A61N 1/04

(52) U.S. Cl. CPC A61N 1/36025; A61N 1/0476; A61N 1/36034

(57) ABSTRACT

Embodiments described herein include a method for electrically stimulating target tissue of a brain using a first array of electrodes and a second array of electrodes different from the first array of electrodes. The first array of electrodes may include a first primary electrode and one or more secondary electrodes, and the second array of electrodes may include a second primary electrode and one or more secondary electrodes. The method may include causing, during a charging phase, current to flow between the first primary electrode and the second primary electrode, each of the first array of electrodes and the second array of electrodes disposed to one side of a target tissue of the brain such that the current passes through the target tissue of the brain. The method may include causing, during a discharging phase, a majority of a discharge current to pass through non-target tissue of the brain.

### CROSS-REFERENCE TO RELATED APPLICATIONS

This application claims priority to and the benefit of U.S. Provisional Application No. 63/711,028, filed Oct. 23, 2024 and titled “Systems and Methods for Pseudomonophasic Brain Stimulation,” the disclosure of which is incorporated by reference herein in its entirety.

### TECHNICAL FIELD

Embodiments herein relate to systems, devices, and methods for stimulating brain tissue of a user.

### BACKGROUND

Transcranial electrical stimulation (TES) is a technique used to modulate brain activity and has potential for providing treatments for a variety of neurological conditions. Known approaches to transcranial electrical stimulation use either monophasic stimulation pulses, in cases when the electrodes are located outside the skin and can be replaced periodically after degradation and damage, or biphasic stimulation pulses when the electrodes are located in the subgaleal space (i.e., under the skin and above the skull). Biphasic stimulation pulses are effective at minimizing degradation of electrodes that can occur with monophasic stimulation; however, biphasic stimulation pulses result in relatively poor neuromodulation effects.

### SUMMARY

In some embodiments, a method for electrically stimulating target tissue of a brain uses at least a first array of electrodes and a second array of electrodes different from the first array of electrodes. The first array of electrodes includes a first primary electrode and one or more secondary electrodes, and the second array of electrodes includes a second primary electrode and one or more secondary electrodes. The method includes causing, during a charging phase, a current to flow between the first primary electrode and the second primary electrode, each of the first array of electrodes and the second array of electrodes disposed to a side of, beside, and/or in close proximity to a target tissue of the brain such that current passes through the target tissue of the brain; and causing, during a discharging phase, a majority of a discharge current to pass through non-target tissue of the brain, where the discharge current flows between at least one of: (i) two or more electrodes within the first array of electrodes, (ii) two or more electrodes within the second array of electrodes, (iii) at least one electrode from the first array of electrodes and a return electrode separate from the first array of electrodes, (iv) at least one electrode from the second array of electrodes and a return electrode separate from the second array of electrodes.

### BRIEF DESCRIPTION OF THE DRAWINGS

FIG. 1 is a schematic block diagram of a brain stimulation system, according to embodiments.

FIG. 2 is a schematic block diagram of a brain stimulation system including two or more electrode arrays, according to embodiments.

FIG. 3A is a flow chart diagram of an example method for providing pseudomonophasic brain stimulation to a user, according to embodiments.

FIG. 3B is a flow chart diagram of an example method for providing interleaved pseudomonophasic brain stimulation to a user, according to embodiments.

FIG. 3C is a flow chart diagram of an example method for providing concurrent pseudomonophasic brain stimulation to a user, according to embodiments.

FIG. 3D is a flow chart diagram of an example method for providing pseudomonophasic brain stimulation to a user, according to embodiments.

FIG. 4A shows one example of electrodes grouped into three arrays, where the distance between electrodes within each array is less than the distance between the arrays.

FIG. 4B shows another example of electrodes grouped into arrays.

FIG. 5A shows a brain stimulation system applying biphasic current pulses, according to embodiments.

FIG. 5B is a graph of an electric field magnitude across target brain tissue when biphasic current pulses are applied, according to embodiments.

FIG. 5C is a graph of membrane potential at an axon hillock of a neuron during application of biphasic current pulses, according to embodiments.

FIG. 5D is a graph showing the low-pass filter properties of neurons.

FIG. 6A shows a brain stimulation system applying pseudomonophasic current pulses, according to embodiments.

FIG. 6B is a graph of electric field magnitude across target brain tissue when pseudomonophasic current pulses are applied, according to embodiments.

FIG. 6C is a graph of membrane potential at an axon hillock of a neuron during application of pseudomonophasic current pulses, according to embodiments.

FIG. 7A shows distributed current flow when an electrode discharges current to a discharge partner electrode that spans a large area.

FIG. 7B shows distributed current flow when an electrode discharges current to a plurality of discharge partner electrodes spaced along an array.

FIG. 7C shows local, compact current flow when a nearby discharge partner electrode is used to discharge current.

FIG. 8A is a schematic diagram of a shallow stimulation target and a deep stimulation target.

FIGS. 8B-8C are graphs of the electric field magnitude across the target tissue as a function of the distance/spacing between the stimulating electrode and the discharge partner electrode(s) at a shallow stimulation target and a deep stimulation target, respectively.

FIG. 9A shows a brain stimulation protocol where stimulation is applied to target brain tissue by applying current between a plurality of pairs of electrodes sequentially over time.

FIG. 9B shows the electric field magnitude across target brain tissue in the time domain and the frequency domain when biphasic stimulation is applied.

FIG. 9C shows the electric field magnitude across target brain tissue in the time domain when pseudomonophasic stimulation is applied with one current source.

FIG. 9D shows the electric field magnitude across target brain tissue in the time domain and the frequency domain when pseudomonophasic stimulation is applied with two current sources.

FIG. 10A is a schematic diagram of a brain stimulation system including a first electrode array and a second electrode array disposed on either side of target brain tissue, according to embodiments.

FIG. 10B shows a diagram of global stimulation between primary electrodes of the first electrode array and the second electrode array of FIG. 10A, and local stimulation between the primary electrode of the first electrode array and a secondary electrode of the first electrode array, of FIG. 10A.

FIG. 10C shows different examples of charging and discharging current pulses.

FIG. 11A shows a brain stimulation system including a first electrode array and a second electrode array in a charge phase (top) and an equivalent circuit diagram of the brain stimulation system in the charge phase (bottom).

FIGS. 11B-11F show different discharging techniques for the brain stimulation system (top row) and circuit diagrams of the discharging techniques (bottom row).

FIG. 12A shows a method of providing interleaved pseudomonophasic brain stimulation using one current source, according to embodiments.

FIG. 12B shows a method for providing concurrent pseudomonophasic brain stimulation using one current source, according to embodiments.

FIG. 13A shows a method for providing interleaved pseudomonophasic brain stimulation using two current sources, according to embodiments.

FIG. 13B shows a method for providing concurrent pseudomonophasic brain stimulation using two current sources, according to embodiments.

FIG. 14A shows a method for providing interleaved pseudomonophasic brain stimulation using three current sources, according to embodiments.

FIG. 14B shows a method for providing concurrent pseudomonophasic brain stimulation using three current sources, according to embodiments.

FIG. 15A shows a method for continuously charging secondary electrodes of a brain stimulation system, according to embodiments.

FIG. 15B shows a method for charging, then discharging, secondary electrodes of a brain stimulation system, according to embodiments.

FIGS. 16A-16D show different designs for secondary electrodes of a brain stimulation system, according to embodiments.

### DETAILED DESCRIPTION

Transcranial electrical stimulation (TES) (e.g., transcranial direct current stimulation tDCS, transcranial alternating current stimulation tACS, etc.) is a method for modulating activity in the brain that can be employed minimally-invasively using electrodes disposed under the skin and/or non-invasively using electrodes disposed on top of the skin. TES can be used as a means for providing therapy for a variety of indications including Major Depression Disorder (MDD), Obsessive Compulsive Disorder (OCD), Anxiety, Addiction, Post Traumatic Stress Disorder (PTSD), Epilepsy, Multiple Sclerosis (MS), Stroke rehabilitation, Pain, motor disorders such as Parkinson's and Freezing of Gait, Cognition, Memory, Alzheimer's, and treating Brain Tumors.

According to some known techniques, TES is typically applied using scalp electrodes placed on top of the skin. These scalp electrodes contact the skin indirectly through a conductive sponge or gel interface such that TES can be applied in a continuous or phasic manner using either a monophasic (charge-imbalanced) or biphasic (charge-balanced) pulsatile waveform. Monophasic pulsatile waveforms include current pulses of one polarity, whereas biphasic waveforms typically include a current pulse of a first polarity closely followed by a current pulse of a second polarity opposite the first polarity. Monophasic stimulation, while more effective at activating neural tissue, can cause electrode degradation over time due to non-reversable faradic reactions that occur at the electrode-tissue interface. This electrode degradation can significantly limit electrode lifetimes and/or damage surrounding tissue. Electrodes can provide monophasic stimulation for one or more hours, depending on the current applied, before they need to be replaced. Monophasic stimulation pulses may be practical for electrodes placed on the scalp (e.g., cap EEG electrodes, EEG net electrodes, and transcutaneous electrical nerve stimulation (TENS) electrodes) as they can be replaced as needed when the electrode degrades. However, a monophasic approach is not possible with electrodes implanted under the skin.

Frequent electrode replacement is not practical in cases where the electrodes are placed under the skin, due to the inconvenience, cost, and health risk (infection, blood vessel hemorrhage, etc.) involved in the electrode replacement surgery. Therefore, electrodes implanted under the skin (e.g., either above or under the skull) should be designed to last for several years at a minimum. Moreover, monophasic stimulation pulses delivered repeatedly to electrodes under the skin can lead to the formation of reactive species from redox reactions, which is unsafe for the user, as it can damage tissue near the electrode, and can be damaging to the electrodes themselves. Therefore, subscalp TES must apply stimulation that is charge-balanced with a net zero current flow after the stimulation waveform cycle is completed.

Biphasic stimulation pulses minimize or reduce degradation of the electrodes placed under the skin, but can also cause the electric fields to oscillate, and fast oscillations can degrade the neuromodulation efficacy due to the low pass filtering properties of neural membranes. In other words, the faster the electric field oscillates, the lower the likelihood that the electric field will activate neurons. Therefore, while biphasic stimulation may increase longevity and safety of TES systems, it can also simultaneously reduce the potency of neuromodulation, especially for short-pulsed stimulation.

Temporal Interference (TI) is another existing approach to transcranial electrical stimulation whereby two high frequency stimuli (typically in the kilohertz (kHz) range) are delivered to two electrodes with a small frequency difference. For example, a stimulus at 1000 Hz may be delivered to a first electrode and a stimulus at 1010 Hz may be delivered to a second electrode to create a low “beat” frequency (e.g., 10 Hz) interference signal where the two signals overlap in the brain, referred to as a Russian Waveform. Advantages of temporal interference are that patients generally tolerate this approach well, there is some cell selectivity, and stimulating current can be spatially steered. However, the stimulation sources used in temporal interference are very high frequency (e.g., on the order of kHz), and the waveforms are inherently biphasic (typically sinusoidal), making them far less potent for neuromodulation than low frequency stimulation (e.g., on the order of hertz). The waveforms created in the brain by multi-electrode temporal interference can also be pre-mixed and applied through a single electrode pair instead of using the Russian Waveform. This pre-mixed approach has been used with TENS (Transcranial Electric Nerve Stimulation) and EMS (electrical Muscle Stimulation). However, delivering the pre-mixed signal to one electrode limits the ability to spatially steer stimulation, which is important when stimulating specific regions of the brain.

The present invention addresses the foregoing drawbacks of known TES systems by preserving electrode longevity and tissue health through the delivery of higher-frequency biphasic pulses in which the second phase is modified so that the signal effectively has characteristics of low-frequency stimulation fields to the brain. In some embodiments, systems and devices described herein deliver a first stimulation pulse between a first set of electrodes that span (traverse) the target (e.g., the neuromodulation target) in the brain, and deliver a second charge recovery pulse of the opposite polarity between a second set of electrodes which do not span the brain target and which are proximal to one another relative to the distance between the second set of electrodes and the brain target. This type of charge balanced stimulation is referred to as ‘pseudo-monophasic stimulation’ from the perspective of the brain target to be modulated.

The embodiments described herein include a method for delivering pseudomonophasic electrical stimuli to the brain using an intersectional stimulation strategy in a way that both maximizes the neuromodulatory strength while maintaining charge-balanced stimulation at the electrode-tissue interface for safety and longevity. This intersectional stimulation strategy is particularly relevant for subgaleal/subscalp transcranial electrical stimulation (i.e., under the scalp but above the skull) and for epidural and subdural electrical stimulation (i.e., under the skull but above the brain surface) where electrode longevity is critical for chronic therapeutic applications. Embodiments described herein include (i) specific types of stimulation waveforms across electrodes to create a monophasic-like stimulation waveform at the brain target, and (ii) examples of hardware circuitry to implement and enable these types of stimulation waveforms.

One or more embodiments described herein provide improved circuitry for transcranial electric stimulation. Additionally, embodiments described herein can improve stimulation effectiveness for intracranial stimulation approaches in which the distance between the electrode lead and the target brain tissue is far relative to the inter-contact distance on the electrode lead. The embodiments described herein can increase longevity of electrodes implanted under the skin while maintaining effectiveness of stimulation, as the present invention allows for the delivery of pseudomonophasic stimulation to the brain target while minimizing the electrode degradation and tissue damage near the electrode. The pseudomonophasic stimulation method results in significantly higher neuromodulation efficacy compared to the traditional biphasic simulation waveforms, while maintaining the electrode safety, stability, and longevity.

One or more embodiments described herein use a combination of electrode design and interleaved stimulation current pulsing to restore the low-frequency content of the composite stimulation waveform which is reduced or eliminated when charge-balancing with biphasic stimulation waveforms. Charge-balanced stimulation is preserved at the electrode-tissue interface for safety, and the combination of electrode design and interleaved intersectional pulsing is used to decouple the modulatory phase of stimulation (e.g., the charging phase) from the charge recovery phase of stimulation (e.g., the discharging phase). The pseudo-monophasic stimulation waveform approach continues to use charge-balanced stimulation pulses from the perspective of the local electrodes but delivers monophasic-like stimulation pulses to the target brain region to be modulated. These pseudo-monophasic stimulation pulses have a large low-frequency component and are therefore more effective at modulating the activity of the neurons in the target brain region.

Embodiments set forth herein leverage and build on existing tDCS, tACS, and intersectional short pulse (ISP) stimulation methods, but improve on these methods by, for example, only permitting stimulation of the target brain tissue during the first phase of the stimulus pulse, and redirecting a majority of the current away from the brain target during the second charge balancing phase.

As used herein, “voltage gradient” refers to the potential difference between two points divided by the distance therebetween. The voltage gradient is related to the electric field by the equation: E=−∇(V). |E| is the magnitude of the electric field.

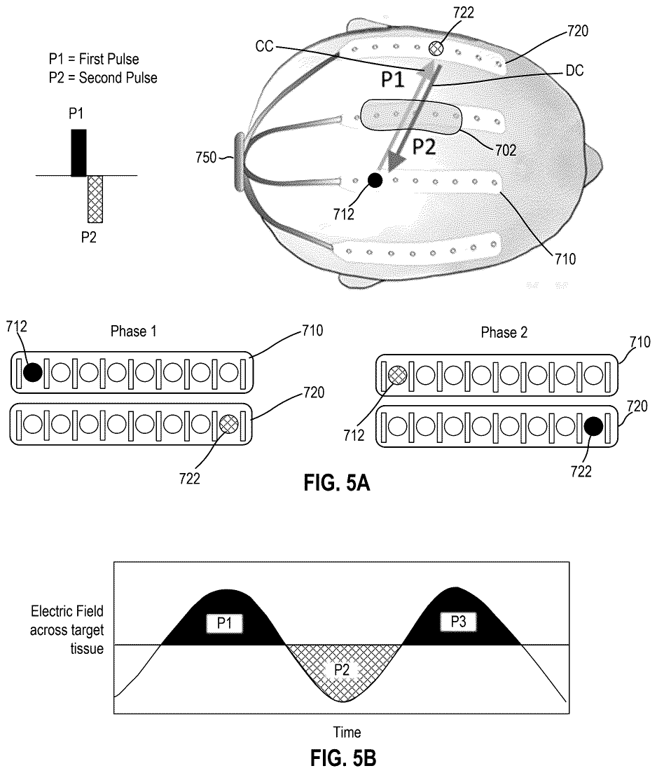

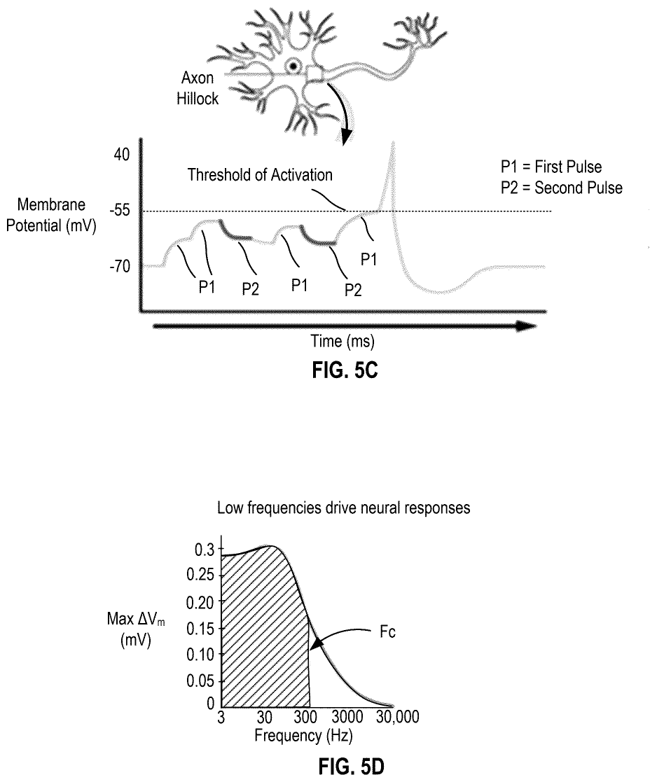

As used herein, “electrode array” refers to a set of electrodes. In some embodiments, the set of electrodes in an electrode array may be clustered or grouped together spatially. For example, neighboring or adjacent electrodes in an electrode array may have a distance therebetween (i.e., an inter-electrode distance) that is smaller than a distance between the electrode array and a neighboring electrode array. Where multiple electrode arrays are present (e.g., as pictured in FIG. 5A), the distances between neighboring electrode arrays can vary (e.g., a distance between a first electrode array from the multiple electrode arrays can be a first distance away from a second electrode array from the multiple electrode arrays, and a distance between a third electrode array from the multiple electrode arrays can be a second distance away from the second electrode array from the multiple electrode arrays, the second distance being different from the first distance) or the distances between neighboring electrode arrays can be substantially consistent. Alternatively or in addition, each set of electrodes in an individual electrode array can have a uniform inter-electrode distance (with respect to the electrodes thereof), or the inter-electrode distance thereof can vary. For example, regarding the latter, a distance between a first electrode from the set of electrodes and a second electrode from the set of electrodes can be a first distance, and a distance between a third electrode from the set of electrodes and the second electrode from the set of electrodes can be a second distance different from the first distance, etc. The set of electrodes in an electrode array may have any suitable spatial organization such as, for example, a linear (or other arrayed) configuration, a clustered configuration, a circular configuration, a grid configuration, etc.

As used herein, “global stimulation” refers to flow of current between two or more electrodes across two or more electrode arrays. For example, global stimulation occurs when current flows between an electrode from a first electrode array and an electrode from a second electrode array. As used herein, “local stimulation” refers to a flow of current between two or more electrodes within an electrode array or between electrodes within an electrode array and a return electrode that is distinct from but near the electrode array. Current that passes through two or more electrodes within an electrode array or between electrodes within an electrode array and a return electrode (e.g., a return electrode near the electrode array) is referred to as “local current.” Local current can also pass internally through wires to a return electrode.

As used herein “primary electrode” refers to electrodes configured to pass current across electrode arrays and within electrode arrays, and therefore are configured for global stimulation and local stimulation. As used herein, “secondary electrodes” or “auxiliary electrodes” are electrodes configured to pass current within an electrode array or from the electrode array to a return electrode or ground, and therefore are configured only for local stimulation. In some embodiments, primary electrodes may be configured to pass current to primary electrodes on different electrode arrays, to secondary electrodes on the same electrode array, to return electrodes separate from the electrode arrays, and/or to ground. In some embodiments, secondary electrodes may be configured to pass current to primary electrodes on the same electrode array, to return electrodes separate from the electrode arrays, and/or to ground.

As used herein, “current waveform” refers to current that passes through any one electrode over time. Each current waveform can have two or more phases (e.g., periods of time, amounts of time, etc.). The “charging phase” or “charge phase” can refer to phases of a current waveform that have a first polarity and “discharging phase” can refer to phases of the current waveform that have a second polarity opposite to the first polarity.

As used herein, “active current” refers to current flow generated by an electrolytic cell (i.e., a battery) between at least one positive electrode (the anode) and at least one negative electrode (the cathode). Passive current refers to non-active current flow between one or more electrodes and a return electrode or between one or more electrodes and ground.

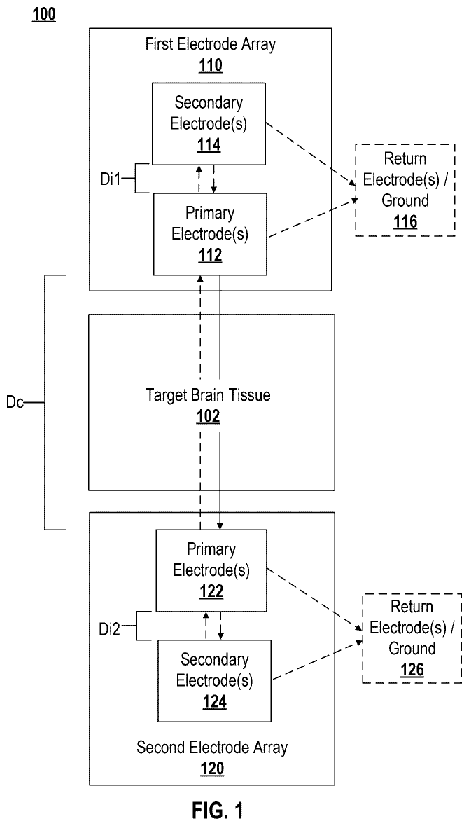

FIG. 1 is a schematic block diagram of a brain stimulation system 100, according to embodiments. In some embodiments, the system 100 may include two or more electrode arrays disposed near a brain of a user or patient. In some embodiments, the two or more electrode arrays may be disposed on a scalp of the user or patient. In some embodiments, the two or more electrode arrays may be disposed under the skin and above the skull, in a subgaleal space. In some embodiments, the two or more electrode arrays may be disposed under the skull, in the subdural space or the epidural space above the surface of the brain.

In some embodiments, the system 100 can include a first electrode and a second electrode disposed near target brain tissue 102 and configured to cause current to flow across or through a target brain tissue 102 during a first phase (e.g., a charging phase). The current during the first phase can have a first polarity configured to cause neural stimulation in the target brain tissue 102. In some embodiments, the first electrode and/or the second electrode can be configured to cause current having a second polarity opposite to the first polarity to flow across or through one or more regions of brain tissue different than the target brain tissue 102 during a second phase (i.e., a discharging phase) to ‘charge balance’ electrode interfaces of each of the first electrode and the second electrode. In some embodiments, during the second phase, the first electrode and the second electrode do not cause current to flow therebetween such that a majority of the discharge current is not directed across or through the target brain tissue 102. The current flow during the first phase and the second phase can be such that a charge at a surface of at least one of the first electrode and the second electrode is zero or near zero after the second phase. Additionally, an electric field or voltage gradient across the target brain tissue 102 is maintained in a first direction during the second phase such that neural stimulation in the target brain tissue 102 is not disrupted, as described in further detail below. In some embodiments, the applied gradient between the first electrode and the second electrode to produce a voltage gradient across the target brain tissue 102 can be maintained within a predetermined/predefined range during the first phase and the second phase. In some embodiments, the predetermined/predefined range of the voltage gradient across the target brain tissue 102 is between about 0.01 V/m to about 1.0 V/ m (inclusive of all values and ranges therebetween) at an applied current of about 1 mA.

In some embodiments, the system 100 may include a first electrode array 110 including one or more primary electrodes 112 (e.g., including the first electrode) and one or more secondary electrodes 114 (also referred to herein as “auxiliary electrodes”). In some embodiments, the system 100 may include one or more return electrodes 116 or a ground separate from the first electrode array 110. The system 100 may further include a second electrode array 120 including one or more primary electrodes 122 (e.g., including the second electrode) and one or more secondary electrodes 124. In some embodiments, the system 100 may include one or more return electrodes 126 or a ground separate from the second array of electrodes 120. In some embodiments, the return electrode(s) 116, 126 may be separate from the first and second electrode arrays 110, 120 but nearby to the first and/or second electrode arrays 110, 120. In some embodiments, the return electrode(s) 116, 126 may be disposed on the first electrode array 110 and/or the second electrode array 120.

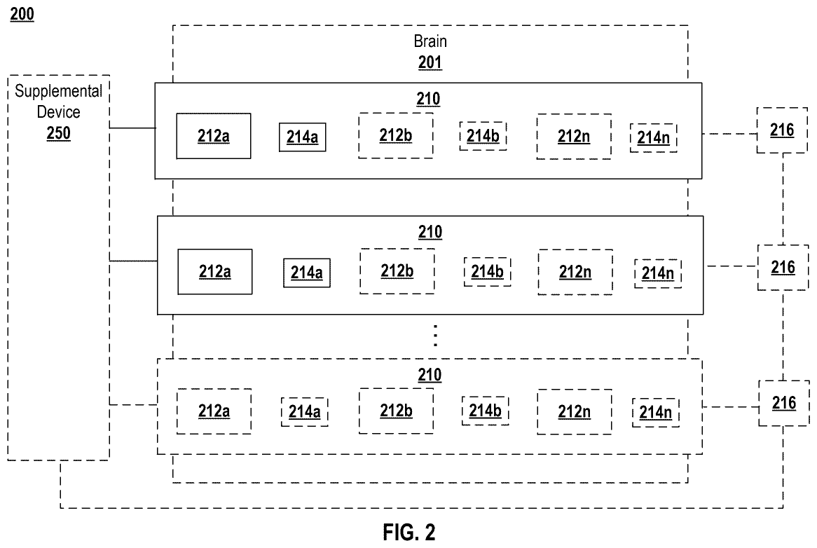

The first electrode array 110 may be disposed adjacent to, beside, to one side of (e.g., to/on a first side of) or in close proximity to the target tissue of the brain (target brain tissue 102) to be modulated, and the second electrode array 120 may be disposed adjacent to or to another side of (e.g., to a second side of) or otherwise in close proximity to the target brain tissue 102 to be modulated. In some embodiments, the system 100 includes a plurality of electrode arrays, each electrode array from the plurality of electrode arrays being disposed over an associated portion of the brain, as described in further detail with respect to FIG. 2.

In some embodiments, the primary electrode(s) 112 of the first electrode array and the primary electrode(s) 122 of the second electrode array may be configured to pass current therebetween. The charging phase can occur when the first current pulse having a first polarity is injected, introduced, or delivered through an electrode in the system for a given stimulation session. Any period of time in which charge with the first polarity is injected, introduced, or delivered through an electrode in the system can be referred to as the charging phase (i.e., charge phase, charging period, charging time). When charge injected, introduced or delivered through electrodes in the system has a second polarity opposite the first polarity, this can be referred to as a discharging phase (i.e., discharge phase, discharging period, discharging time). Charging phases and discharging phases can be repeated in a predefined sequence to generate at least a portion of a waveform for modulating the target brain tissue 102. In some embodiments, the charging phases may predominantly drive neural behavior at the target brain tissue 102 and discharging phases may have small or no impact on neural behavior at the target brain tissue 102. In some embodiments, the current waveform through each electrode is charge balanced, meaning the total charge injected, introduced, or delivered through each electrode (e.g., the current integrated over time) is close to or equal to zero after all phases of stimulation have been completed. Charge balancing the stimulation can prevent electrode degradation and faradaic reactions from occurring in the tissue near the active electrode interface.

In some embodiments, current may be directed from a first primary electrode of the first electrode array 110 to a second primary electrode of the second electrode array 120 such that current flows through the target brain tissue 102 and generates an electric field at the target brain tissue 102 (e.g., global stimulation). In some embodiments, the primary electrodes 112, 122 in each electrode array 110, 120 may be configured as an anode or a cathode based on a location of the target brain tissue 102 to be stimulated and/or whether the target brain tissue 102 is to be excited or inhibited. As current flows between the first primary electrode 112 and the second primary electrode 122, charge can build up at the surfaces of the first and/or second primary electrodes 112, 122. For example, a first charge can build up on a first electrode interface of the first primary electrode 112 and a second charge can build up on a second electrode interface of the second primary electrode 122. In such instances, discharging may be performed to reduce the charge build-up to zero or close to zero. In some embodiments, each of the first electrode array 110 and the second electrode array 120 may include a plurality of primary electrodes 112, 122 and current may be configured to flow between at least one of the primary electrodes 112 of the first electrode array 110 to the plurality of primary electrodes 122 of the second electrode array 120 (and vice versa). For example, current may flow from one electrode in an electrode array to N electrodes in a different electrode array. Conversely current may flow from N electrodes in an electrode array to one electrode in a different electrode array. In some embodiments, current may flow from N electrodes in an electrode array to M electrodes in a different electrode array.

In some embodiments, during the discharging phase, discharge current can be directed between the first electrode that was previously activated (e.g., the first primary electrode 112) and one or more discharge partner electrodes. The discharge partner electrodes can be positioned such that the discharge current is not directed or steered through the target brain tissue 102. The discharge partner electrodes can include one or more electrodes within or coupled to the first electrode array 110 such as one or more secondary electrodes 114 or one or more return electrodes 116. The discharge partner electrodes of the first primary electrode 112 can be disposed near the first primary electrode 112 such that the discharge current only affects a small region of brain tissue. In some embodiments, during the charging phase, charge can be directed between the second electrode that was previously activated (e.g., the second primary electrode 122) and one or more discharge partner electrodes. The one or more discharge partner electrodes of the second primary electrode 122 can include one or more electrodes within or coupled to the second electrode array 120 such as one or more secondary electrodes 124 or one or more return electrodes 126. The one or more discharge partner electrodes of the second primary electrode 122 can be positioned such that the discharge current from the second primary electrode 122 is not directed or steered through the target brain tissue 102. The discharge partner electrodes of the second primary electrode 122 can be disposed near the second primary electrode such that the discharge current only affects a small region of brain tissue, or does not substantially affect the brain tissue.

In some embodiments, the discharge partner electrode of the first primary electrode 112 can include a primary electrode that is different than the primary electrode activated during the previous charging phase (e.g., the second primary electrode 122). For example, the first primary electrode 112 can discharge to a different primary electrode, and this electrode pair may be positioned to cause a desired modulatory effect (e.g., stimulation or inhibition) of a different target brain tissue than the target brain tissue 102. In some embodiments, a different set of electrode(s) from the first primary electrode 112 and the second primary electrode 122 can be activated during the discharge phase. Therefore, modulation can occur during discharging of the first primary electrode 112. Similarly, the discharge partner electrode of the second primary electrode 122 can include a primary electrode that is different than the first primary electrode 112.

In some embodiments, during a discharging phase, a discharge current can be directed between (i) two or more electrodes within the first array of electrodes (e.g., active current); (ii) two or more electrodes within the second array of electrodes 120 (e.g., active current); (iii) at least one electrode from the first array of electrodes 110 and a return electrode separate from the first array of electrodes (e.g., passive current); and/or (iv) at least one electrode from the second array of electrodes and a return electrode separate from the second array of electrodes (e.g., passive current). In some embodiments, during the discharging phase, current may flow from at least one electrode in the first electrode array to the return electrode through a wire. In some embodiments, current may flow from at least one electrode in the second electrode array to the return electrode 126 through a wire. In some embodiments, performing at least one of (i) or (iii) can generate a third charge at the first electrode interface equal in magnitude and opposite in polarity to the first charge at the first electrode interface of the first primary electrode 112. Therefore, performing at least one of (i) or (iii) can balance the charge on the first electrode interface. In some embodiments, performing at least one of (ii) or (iv) can generate a fourth charge at the second electrode interface equal in magnitude and opposite in polarity to the second charge at the second electrode interface of the second primary electrode 122. Therefore, performing at least one of (ii) or (iv) can balance the charge on the second electrode interface. In some embodiments, all of the charging phases occur actively, and all of the discharging phases occur passively. In some embodiments, all of the charging phases occur actively, and all the discharging phases occur actively. In some embodiments, all of the charging phases occur actively, and the discharging phases are a combination of active and passive.

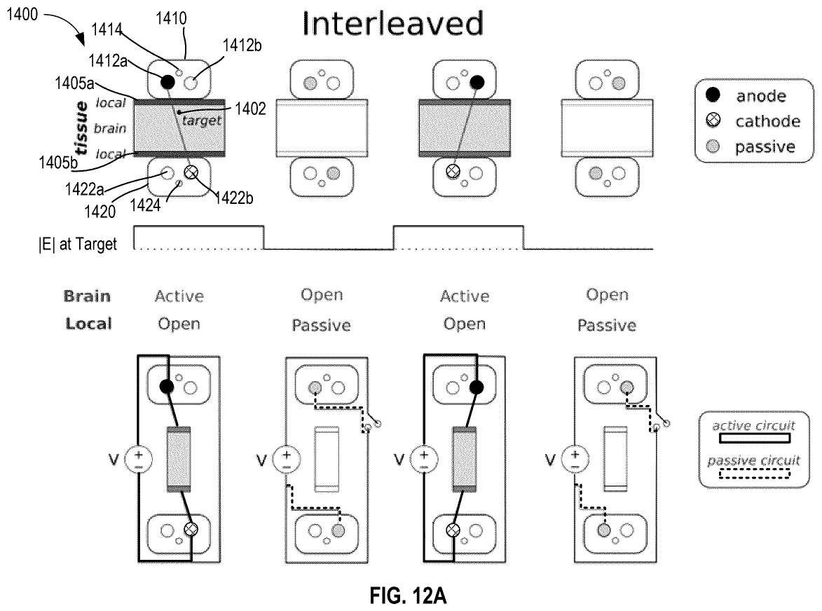

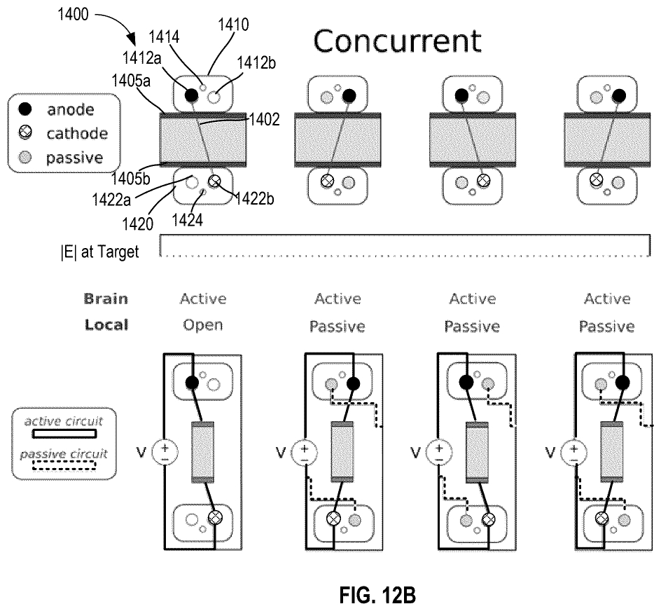

In some embodiments, if during the charging phase, the first primary electrode is configured as an anode and the second primary electrode is configured as a cathode such that current flows from the first primary electrode 112, through the target brain tissue 102, and to the second primary electrode 122, then during the discharging phase, current may be directed from the first primary electrode 112 to the secondary electrode 114 of the first electrode array 110. Alternatively or additionally, current may be caused to flow between the first primary electrode 112 and the return electrode 116 and/or between the secondary electrode 114 of the first electrode array 110 and the return electrode 116 and/or to ground. Additionally or alternatively, current may be caused to flow between the second primary electrode 122 and the return electrode 126 (or ground) and/or between the secondary electrode of the second electrode array 120 and the return electrode 126 and/or to ground. In some embodiments, stimulation may be performed with one current source, two current sources, and/or three current sources, as described in further detail below with reference to FIGS. 12A-14B.

A within-array distance (e.g., “Di1,” “Di2”) is defined as a distance between edges of any two electrodes within the same array. In some embodiments, electrodes across arrays may have an across-array distance Dc therebetween. The across-array distance Dc is defined as a distance between edges of an electrode of a first electrode array and edges an electrode of a second electrode array. In some embodiments, the within-array distances Di1, Di2 may be less than the across-array distances Dc. In some embodiments, electrodes within an array may have a within-array distance below a predetermined threshold such that discharge current is localized over a small area of brain tissue. In some embodiments, the within-array distance(s) Di1, Di2 may vary based on a depth of the target brain tissue for stimulation. For example, for target brain tissue that is deeper in the brain, the within-array distance Di1, Di2 may be larger than for target brain tissue that is shallower in the brain. Small within-array distances Di1, Di2 prevent discharge current from distributing over a large area, thereby preventing disturbance of a larger area of brain tissue. In some embodiments, a distance between the electrode arrays and the target brain tissue 102 may be greater than a within-array distance. In some embodiments, the distance between the electrode arrays and the target brain tissue 102 may be 2 times greater than a within-array distance.

In some embodiments, a distance between the edges any two adjacent pairs of electrodes (primary or secondary) within an array is less than 0.5 cm, less than 1 cm, less than 1.5 cm, less than 2 cm, less than 2.5 cm, less than 3 cm, less than 3.5 cm, less than 4 cm. In some embodiments, the distance between the edges any two adjacent pairs of electrodes (primary or secondary) within an array two electrodes is less than 1 cm. In some embodiments, a shortest across-array distance may be greater than 1 cm, greater than 2 cm, greater than 3 cm, greater than 4 cm, greater than 5 cm, greater than 6 cm, greater than 7 cm, greater than 8 cm, greater than 9 cm, greater than 10 cm. In some embodiments, a ratio of the across-array distance to a within-array distance is greater than or equal to 2.

In some embodiments, the brain stimulation system 100 may be configured to deliver interleaved stimulation in which a plurality of charging phases and discharging phases are repeated sequentially, meaning no charging phase from the plurality of charging phases overlaps in time with any other charging or discharging phase and/or no discharging phase from the plurality of discharging phases overlaps in time with any other charging or discharging phase. In some embodiments, the brain stimulation system 100 may be configured to deliver concurrent stimulation in which at least some charging and discharging phases can overlap in time and/or occur simultaneously. For example, during concurrent stimulation, a discharging phase of the first primary electrode 112 can be concurrent with a charging phase of the second primary electrode 122, and vice versa. In some embodiments, the brain stimulation system 100 can deliver interleaved stimulation with active discharge phases. In some embodiments, the brain stimulation system can deliver interleaved stimulation with passive discharge phases. In some embodiments, the brain stimulation system 100 can deliver concurrent stimulation (e.g., temporally overlapping waveforms) with active discharge phases. In some embodiments, the brain stimulation system 100 can deliver concurrent stimulation with passive discharge phases.

In some embodiments, the charging phase(s) may have a duration in a range of about 0.1 millisecond (ms) to about 500 ms, inclusive of all ranges and subranges therebetween. In some embodiments, the discharging phases may have a duration in a range of about 0.1 ms to about 500 ms, inclusive of all ranges and subranges therebetween. In some embodiments, a maximum duration of a single phase (e.g., charging phase and/or discharging phase) of a stimulation waveform is less than 1000 ms, less than 900 ms, less than 800 ms, less than 700 ms, less than 600 ms, less than 500 ms, less than 400 ms, less than 300 ms, less than 200 ms, less than 100 ms. In some embodiments, the maximum duration of a single phase of a stimulation waveform is less than 500 ms. In some embodiments, the stimulation waveform generated when repeating the charging and discharging phases may have a frequency that is less than about 250 Hertz (Hz). In some embodiments, the frequency of the stimulation waveform may be below a cut-off frequency of a typical neuron (e.g., around approximately 250 Hz to 500 Hz).

FIG. 2 is a schematic block diagram of a brain stimulation system 200 including two or more electrode arrays 210 disposed near a brain 201 of a user (e.g., a patient). In some embodiments, the brain stimulation system 200 may include a plurality of electrode arrays 210 implanted under the skin or under the skull and configured to cover a respective portion of the brain 201. In some embodiments, the system 110 may include a number of electrode arrays in a range of 2 electrode arrays to 100 electrode arrays, inclusive of any ranges or subranges therebetween. In some embodiments, each electrode array may be configured to be positioned around a periphery of a respective target region of the brain to be stimulated.

In some embodiments, the electrode arrays 210 may each include one or more primary electrodes 212a, 212b, . . . 212n and one or more secondary electrodes 214a, 214b, . . . 214n. In some embodiments, each electrode array 210 may include any suitable number of primary electrodes 212a-212n and/or secondary electrodes 214a-214n. In some embodiments, the electrode arrays 210 may each include between about 1 primary electrode and about 1000 primary electrodes, inclusive of all ranges and subranges therebetween. In some embodiments, the electrode arrays 210 may each include between about 1 secondary electrode and about 1000 secondary electrodes, inclusive of all ranges and subranges therebetween. In some embodiments, each electrode array 210 may be coupled to one or more return electrodes 216 separate from each electrode array 210. In some embodiments, the return electrodes 216 may provide a route for discharging current from the primary electrodes 212a-212n and/or secondary electrodes 214a-214n. In some embodiments, the electrode arrays 210 may be operatively coupled to a supplemental device 250 configured to supply current and/or power. In some embodiments, the supplemental device 250 may be configured to store stimulation protocols such as any of the stimulation protocols described herein.

In some embodiments, the electrode arrays (e.g., electrode leads) may include any suitable electrode contact such as, for example, paddle electrodes, strip electrodes, grid electrodes, needle electrodes, depth electrodes, or a suitable combination thereof. The electrodes may be arranged in any suitable arrangement such as, for example, a linear arrangement, a grid, a lattice, or a suitable combination thereof. In some embodiments, the primary electrodes 112, 122 may be arranged linearly along an electrode lead, and a plurality of electrode leads may be disposed substantially parallel to one another.

In some embodiments, the system 200 can be configured such that during a charging phase, a first set of primary electrodes of the plurality of electrodes 212a-212n are activated such that current is directed or steered through a first target region of the brain. In some embodiments, during a discharging phase, a first set of discharge electrodes can be activated to discharge electrode interfaces of the first set of primary electrodes. In some embodiments, each primary electrode of the first set of primary electrodes can have one or more discharge partner electrodes (e.g., one or more secondary electrodes and/or one or more ground electrodes). The one or more discharge partner electrodes can be configured such that discharge current does not flow through the first target region of the brain. In some embodiments, during the discharging phase, a second set of primary electrodes from the plurality of electrodes 212a-212n can be activated such that charging current is directed through the first target region of the brain (i.e., concurrent stimulation). Therefore, the first target region of the brain can continue to receive stimulation while the first set of primary electrodes is discharged. Alternatively, the second set of primary electrodes can be activated after the discharging phase rather than during the discharging phase (i.e., interleaved stimulation). In some embodiments, a subsequent set of primary electrodes can be activated during each charging phase such that the previously activated set of primary electrodes can be discharged. Interleaving the set of primary electrodes that are activated allows for high efficiency stimulation while also allowing charge to balance at each electrode interface of the primary electrodes following activation. In some embodiments, a plurality of target brain regions can be stimulated simultaneously or during overlapping periods of time.

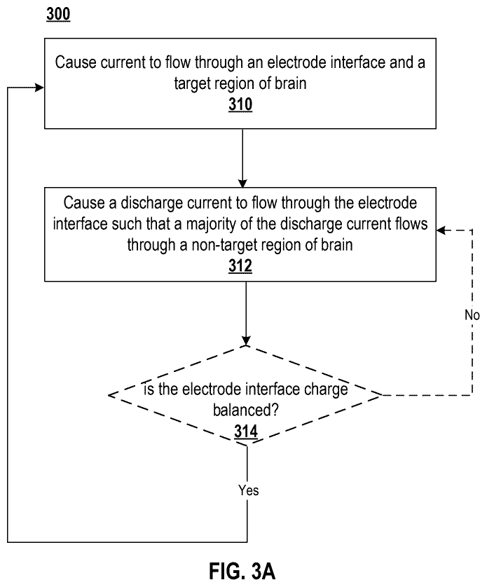

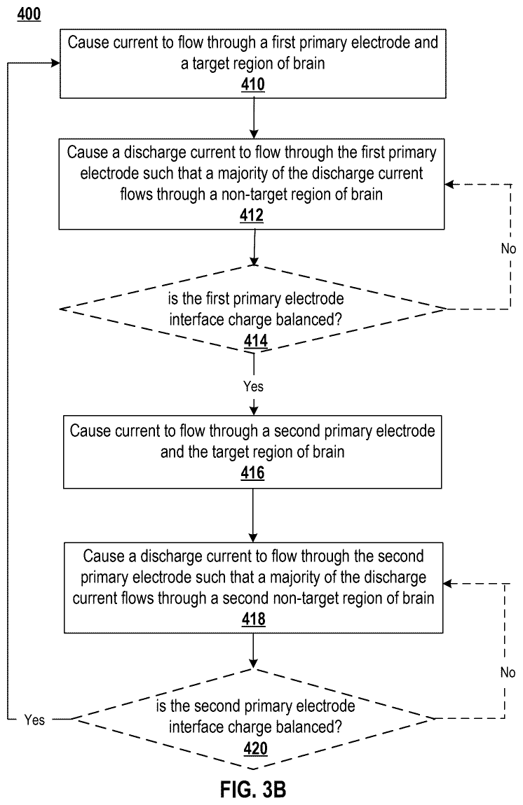

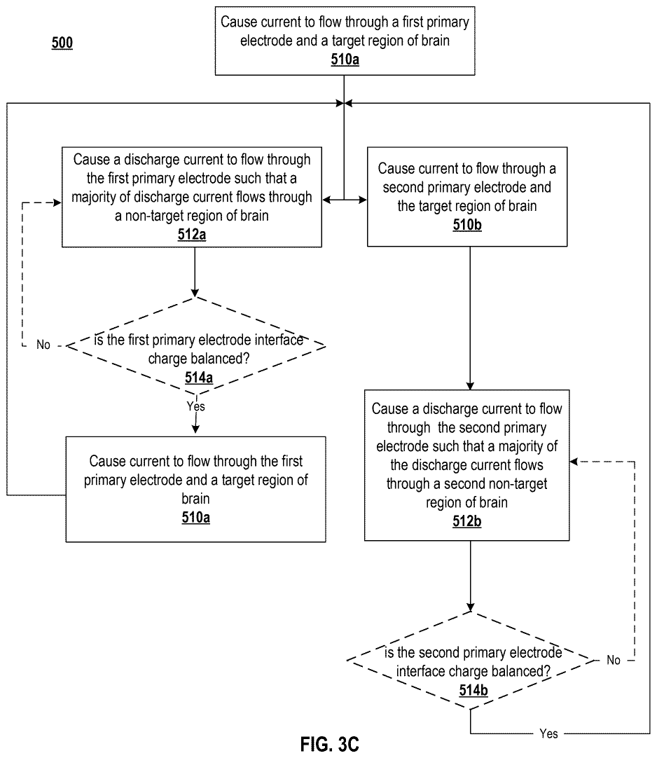

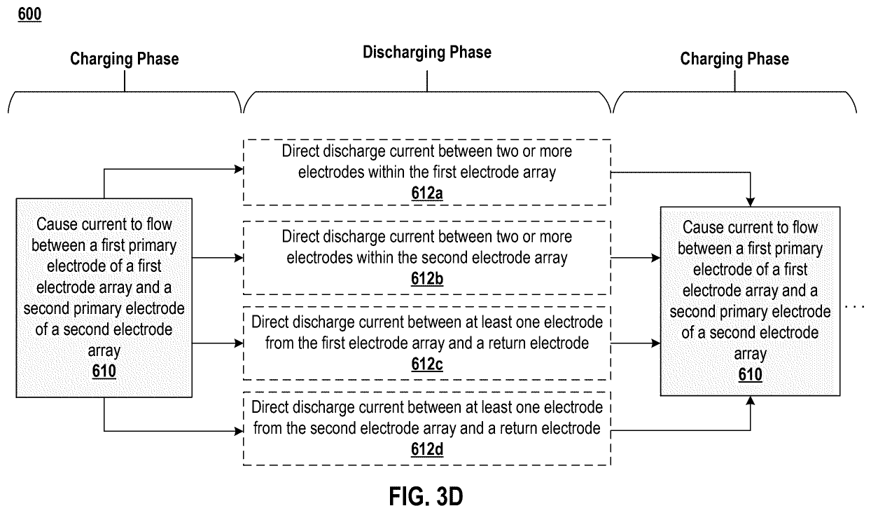

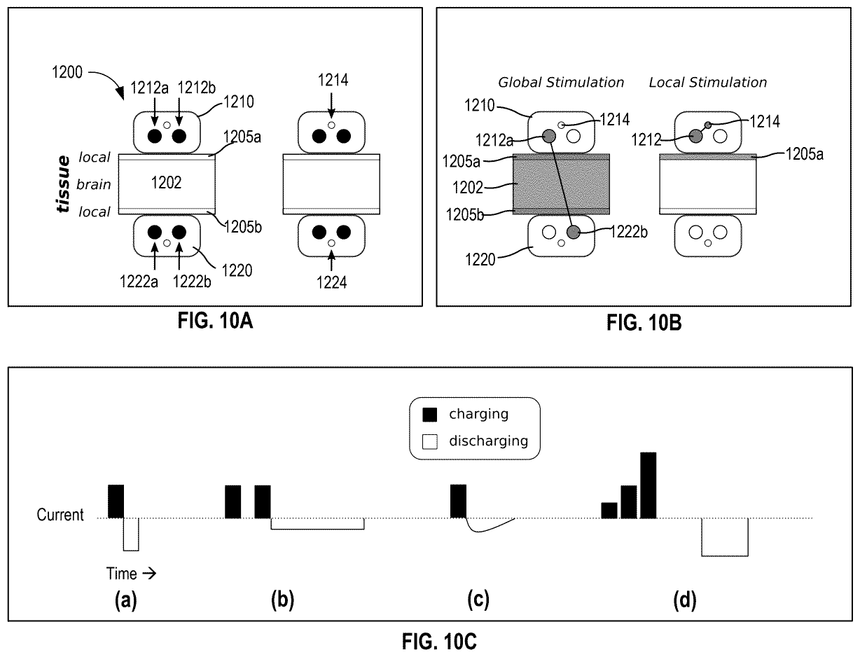

FIG. 3A is a flow chart diagram of an example method 300 for providing pseudomonophasic brain stimulation to a user, according to embodiments. As shown, method 300 includes causing a current to flow through an electrode interface and a target region of the brain. In some embodiments, the causing the current to flow may occur during a first period of time (e.g., corresponding to or overlapping with the charging phase). In some embodiments, the causing the current to flow through the electrode interface may include causing current to flow from one or more primary electrodes of a first electrode array disposed on a first side of the target brain region to one or more primary electrodes of a second electrode array disposed on a second side of the target brain region. The method 300 may further include causing a discharge current (e.g., a current of opposite polarity to the current flowing during the charging phase) to flow through the electrode interface such that a majority (or all) of the discharge current flows through a non-target region of the brain. In some embodiments, the method 300 may include causing the discharge current to flow through the electrode interface without causing current to pass through the target region of the brain. In some embodiments, the causing the discharge current to flow may occur for a second period of time (e.g., a discharging phase). In some embodiments, the non-target region of the brain may be a portion of brain tissue proximate to the electrode interface (e.g., local tissue). In some embodiments, during the discharging phase, the discharge current may flow through the local tissue and not through the target region of the brain such that a voltage gradient or electric field across the brain tissue in the target region of the brain is maintained within a desired range. In some embodiments, discharging the current through local tissue may prevent the voltage gradient or electric field across the target region of the brain from changing direction. In some embodiments, discharging current through local tissue rather than through the target region of the brain may allow slow (e.g., low frequency, such as 0-100 Hz) changes in the electric field across the target region of the brain. In some embodiments, the method may include causing the discharge current to flow through the electrode interface until the electrode interface is charge balanced, at 314. Parameters of the charging phases and the discharging phases can be determined as described below with respect to FIG. 10C. If the electrode is charge balanced, the method 300 may be repeated any number of times to generate a stimulation waveform. In some embodiments, the method 300 may be repeated across a plurality of electrode interfaces. For example, current may be directed through different electrode interfaces with each charge/discharge cycle. In some embodiments, each charge/discharge cycle may target the same target region of the brain. In some embodiments, each charge/discharge cycle may target a different region of the brain.

FIG. 3B is a flow chart diagram of an example method 400 for providing interleaved pseudomonophasic brain stimulation to a user, according to embodiments. The method 400 may include causing current to flow through a first primary electrode (e.g., of a first electrode array) and a target region of the brain, at 410. In some embodiments, the current may flow through the first primary electrode and the target region of the brain for a first period of time. For example, the current may be flowed (e.g., directed, passed, moved, etc.) from the first primary electrode (e.g., of the first electrode array), through the target region of the brain, and to a second primary electrode of a second electrode array (or vice versa) for a first period of time. As the current is directed between the first primary electrode and the second primary electrode, an amount of charge having a first polarity may accumulate on an electrode interface of the first primary electrode. After the first period of time, the method 400 may include causing a discharge current to flow through the first primary electrode such that a majority (or all) of the discharge current flows through a non-target region of the brain, at 412. In some embodiments, the method 400 may include causing the discharge current to flow through the first primary electrode without causing current to pass through the target region of the brain. For example, the discharge current may be directed from the first primary electrode to a secondary electrode in the first electrode array. In some embodiments, the discharge current may be directed through the first primary electrode and the secondary electrode (e.g., of the first electrode array) to a return electrode separate from the first electrode array. In some embodiments, the discharge current may flow from the first primary electrode to ground and the second primary electrode to ground. In some embodiments, the causing the discharge current to flow may occur for a second period of time. The non-target region of the brain may include brain tissue localized near the first electrode array (e.g., “local tissue”). In some embodiments, during the discharging phase, the discharge current may flow through local tissue and not through the target region of the brain such that a voltage gradient or electric field across the target brain tissue is maintained within a desired range or above a predetermined magnitude. In some embodiments, discharging the current through local tissue may prevent the voltage gradient or electric field across the target region of the brain from changing direction. In some embodiments, discharging current through local tissue may allow slow (e.g., low frequency) changes in the electric field across the target region of the brain.

In some embodiments, the magnitude of the discharge current and a duration of the second period of time may be configured to charge balance the current applied over the first period of time such that the electrode interface of the first primary electrode has little or no accumulation of charge. In some embodiments, the magnitude of the discharge current and the duration of the second period of time can be determined according to the equations described below with respect to FIG. 10C. The method 400 may optionally include continuing to cause the discharge current to flow until the first primary electrode interface is charge balanced, at 414. If the first primary electrode interface is not charge balanced, the method 400 may include continuing to cause the discharge current to flow through the first primary electrode and the non-target region of the brain, at 414. If the first primary electrode interface is charge balanced, the method 400 may include causing current to flow through a second primary electrode (e.g., of a second electrode array) and the target region of the brain for a third period of time, at 416. In some embodiments, the target region of the brain may be the same at step 410 and 416. In some embodiments, the target region of the brain may be different at step 410 and 416. For example, the current flowing through the second primary electrode may be configured to flow through a different target region of the brain than the first primary electrode.

After the third period of time, the method 400 may include causing a discharge current to flow through the second primary electrode such that a majority of the discharge current flows through a second non-target region of the brain for a fourth period of time, at 418. In some embodiments, the discharge current may be caused to flow through the second primary electrode without causing current to pass through the target region of the brain. For example, the discharge current may be flowed from the second primary electrode to a secondary electrode (e.g., in the second electrode array). In some embodiments, the discharge current may be flowed from the second primary electrode to a return electrode separate from the second array. The second non-target region of the brain may be tissue nearby the second electrode array. The method 400 may optionally include continuing to cause the discharge current to flow until the second primary electrode interface is charge balanced, at 420. If the second primary electrode interface is not charge balanced, the method 400 may include continuing to cause the discharge current to flow through the second primary electrode and the second non-target region of the brain. If the second primary electrode interface is charge balanced, the method 400 may be repeated. For example, current may be caused to flow through the first primary electrode (e.g., of the first electrode array) and the target region of the brain for a fifth period of time. In some embodiments, the method 400 may be repeated a plurality of times to generate a stimulation waveform in which an electric field is maintained within a predetermined range at the target brain tissue throughout the stimulation waveform, thereby increasing likelihood of activation of neurons in the target brain tissue. In some embodiments, discharging the current through local tissue may prevent the voltage gradient or electric field across the target region of the brain from changing direction. In some embodiments, discharging current through local tissue may allow slow (e.g., low frequency) changes in the electric field across the target region of the brain.

FIG. 3C is a flow chart diagram of an example method 500 for providing concurrent brain stimulation to a user, according to embodiments. The method 500 may include causing current to flow through a first primary electrode (e.g., of a first electrode array) and a target region of the brain, at 510a. In some embodiments, the causing current to flow through the first primary electrode and the target region of the brain may occur for a first period of time. For example, the current may be directed from the first primary electrode, through the target region of the brain, and to a second primary electrode of a second electrode array (or vice versa) for a first period of time. After the first period of time, the method 500 may include causing a discharge current to flow through the first primary electrode such that a majority of the discharge current flows through a non-target region of the brain (or such that current does not pass through the target region of the brain), at 512a. The discharging of the first primary electrode may be substantially similar to that described in FIGS. 3A-3B. In some embodiments, the causing the discharge current to flow through the first primary electrode may occur for a second period of time. In some embodiments, the method 500 may further include causing current to flow through a second primary electrode (e.g., of a second electrode array) and the target region of the brain, at 510b. During concurrent brain stimulation, discharging of the first primary electrode and the charging of the second primary electrode may at least partially overlap in time. In some embodiments, the causing current to flow through the second primary electrode may occur during a third period of time. In some embodiments, the second period of time and the third period of time may be the simultaneous. In some embodiments, the second period of time and the third period of time may not be simultaneous.

The method 500 may optionally include continuing to cause the discharge current to flow until the first primary electrode interface is charge balanced, at 514a. In some embodiments, the charging phase of the second primary electrode may continue until the first primary electrode interface is charge balanced. In some embodiments, the charging phase of the second primary electrode may end before the first primary electrode interface is charge balanced. In some embodiments, the charging phase of the second primary electrode may have a longer duration than the discharging phase of the first primary electrode.

After the second period of time, the method 500 may include causing current to flow through the primary electrode and the target region of brain (e.g., for a fourth period of time), at 510a. After the third period of time, the method 500 may include causing discharge current to flow through the second primary electrode such that a majority of the discharge current flows through a second non-target region of the brain (e.g., for a fifth period of time), at 512b. In some embodiments, the fourth period of time and the fifth period of time may be simultaneous. In some embodiments, the fourth period of time and the fifth period of time may not be simultaneous. In some embodiments, the method 500 may optionally include discharging the discharge current through the second primary electrode and the second non-target region of the brain until the second primary electrode is charge balanced, at 514b. If the second primary electrode is charge balanced, the method 500 may be repeated starting from steps 512a and 510b to generate a stimulation waveform.

FIG. 3D is a flow chart diagram of an example method for providing pseudomonophasic brain stimulation to a user, according to embodiments. In some embodiments, the method 600 may include causing current to flow between a first primary electrode of a first electrode array and a second primary electrode of a second electrode array, at 610, referred to as the charging phase. In some embodiments, the first primary electrode and the second primary electrode may be oriented around a target region of the brain such that when current is flowed therebetween, current flows through the target region of the brain. In some embodiments, to charge balance the stimulation, the method 600 may include a discharging phase. The discharging phase may include directing discharge current between two or more electrodes within the first electrode array, at 612a. For example, discharge current may be caused to flow between the first primary electrode and a secondary electrode in the first electrode array. Additionally or alternatively, the discharging phase may include directing discharge current between two or more electrodes within the second electrode array, at 612b. discharge current may be caused to flow between the second primary electrode and a secondary electrode in the second electrode array. In some embodiments, the discharging phase may include directing discharge current between at least one electrode from the first electrode array and a return electrode, at 612c. For example, the discharge current may be directed from the first primary electrode and/or the secondary electrode of the first electrode array to a first return electrode or to ground. The discharging phase may further include directing discharge current between at least one electrode from the second electrode array and a return electrode, at 612d. For example, the discharge current may be caused to flow between the second primary electrode and/or the secondary electrode of the second electrode array and a return electrode or ground. In some embodiments, during the discharging phase, discharge current flowing through the target brain region may be close to or equal to zero. In some embodiments, the discharge current may be directed to flow through a non-target region of the brain (e.g., local tissue). For example, the discharge current may be flowed between electrodes within an array that are close in distance and/or not spanning over the target region of the brain.

In some embodiments, after the discharging phase, a subsequent charging phase may occur. For example, the method may include causing the current to flow between the first primary electrode and the second primary electrode, at 610. In some embodiments, the charging phase and the discharging phase may be repeated to generate a waveform for stimulating a target region of a brain.

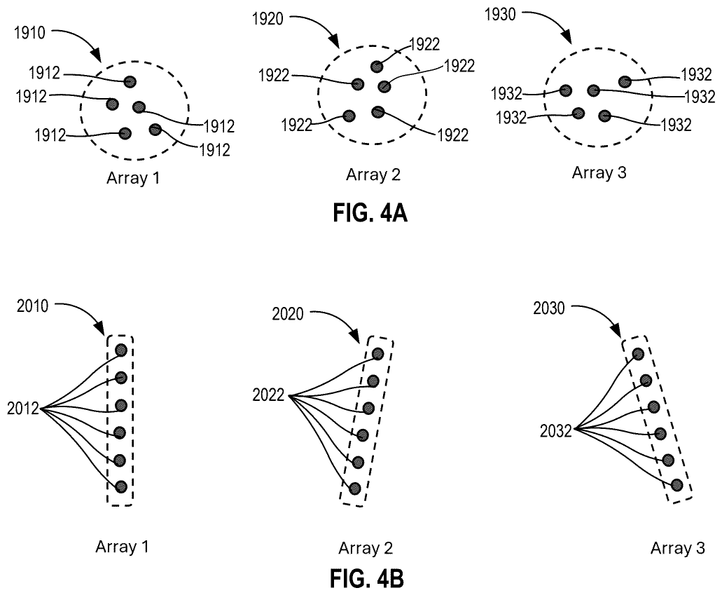

FIG. 4A shows one example of electrodes 1912, 1922, 1932 grouped into three arrays 1910, 1920, 1930. As shown, the spatial distribution of electrodes 1912, 1922, 1932 on the skull can be arranged such that there are three groupings or clusters of electrodes. In some embodiments, a distance between electrodes 1912, 1922, 1932 within each electrode array 1910, 1920, 1930 is less than the distance between electrodes across the electrode arrays 1910, 1920, 1930. The electrode arrays may have any suitable spatial configuration. For example, FIG. 4B shows another example of electrodes grouped into arrays 2010, 2020, 2030. As shown, the electrodes 2012, 2022, 2032 of each array 2010, 2020, 2030 are arranged in a linear configuration.

FIG. 5A shows a brain stimulation system, including electrode arrays 710, 720 coupled to supplemental device 750, applying biphasic current pulses, according to embodiments. Electrical stimulation to electrodes traditionally uses biphasic pulses (e.g., current of one polarity followed by a second pulse of opposite polarity in a fashion where the total net electrical charge transferred after the completion of both pulses is zero) to prevent faradaic reactions at the electrode-tissue interface which degrade and destroy the electrode over time and cause tissue damage. As shown in FIG. 5A, a first current pulse P1 can be directed from a first primary electrode 712 of a first electrode array (e.g., the anode) to a second primary electrode 722 of a second electrode array (e.g., the cathode) such that a charging current CC flows through target tissue of the brain 702. Then, a second pulse P2 can be directed from the second primary electrode 722 (e.g., now the anode) to the first primary electrode 712 (e.g., now the cathode) such that a discharge current DC flows through the target tissue of the brain 702 and through the electrode interfaces of the first and second primary electrodes 712, 722. As shown, the second pulse P2 is configured to balance the charge at a surface of the first primary electrode 712 and the charge at a surface of the second primary electrode 722. One drawback of this method is that biphasic alternating current stimulation can be inefficient at achieving the desired voltage gradient (e.g., 1 V/m) across the target brain region 702 to strongly modulate the activity. FIG. 5B is a diagram showing the electric field (e.g., the magnitude of the electric field) of target brain tissue 702 when biphasic current pulses P1, P2 are applied. As shown, the electric field at the target tissue fluctuates across zero such that the electric field generated during the first current pulse P1 is counteracted by the electric field generated during the second current pulse. For this reason, biphasic current pulses can be inefficient at stimulating brain tissue.

FIG. 5C is a graph of membrane potential at an axon hillock of a neuron during application of biphasic current pulses, according to embodiments. With TES, the goal is to drive the resting membrane potential up or down to the maximal extent possible, biasing the firing probabilities so that neurons are more or less likely to fire, respectively. For example, in some embodiments, if the goal is to activate a neuron and create an action potential, the membrane potential can be driven in the positive direction toward the threshold of activation (e.g., between about −40 millivolts (mV) to about −65 mV). There is a temporal window of tens to hundreds of milliseconds over which the integration of the stimulation pulses has an effect. Biphasic stimulation across the target brain region to be modulated has a negative, counter effect in that the second pulse (opposite in polarity from the first) drives the neural membrane potential in the opposite direction of the first pulse, and therefore reduces the efficacy of the pulses given in rapid succession to drive the neuron towards the firing threshold, as shown in FIG. 5C. Therefore, the two phases of a fast biphasic waveform counteract each other before the membrane has time to respond to either phase of the stimulus, so these biphasic pulses in rapid succession can be inefficient at driving the neuron's membrane potential in any direction.

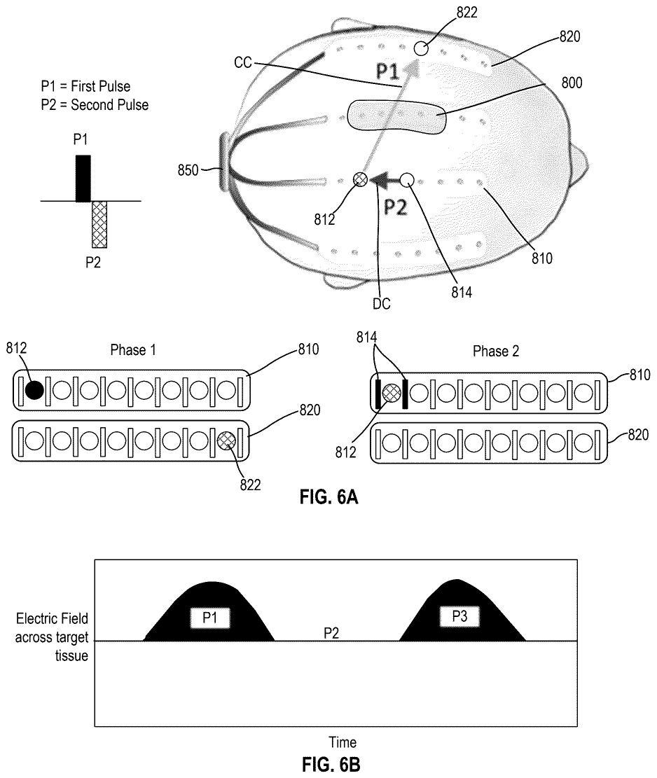

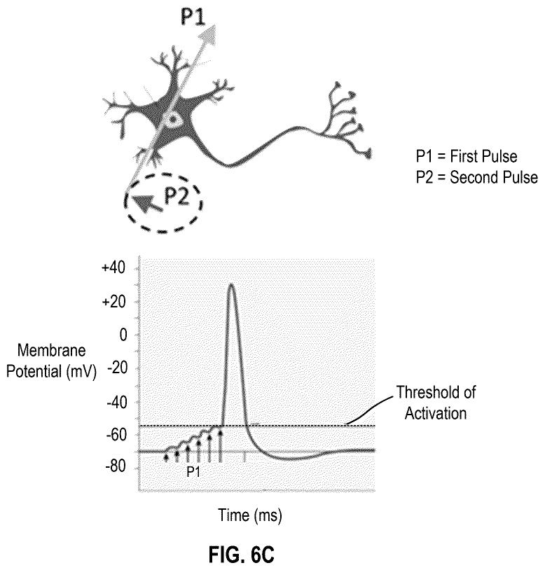

In contrast, monophasic pulses with only one polarity more quickly drive the neuron at the target towards the action potential firing threshold, described in further detail with respect to FIG. 6D. Monophasic pulses with only one polarity are more efficient at driving the target neurons towards (or away from) the action potential firing threshold as illustrated below.

Due to the firing properties of neurons, neurons act as low-pass filters with regard to their input-output transfer function, as shown in FIG. 5D. Therefore, neurons in the brain respond most strongly (i.e., their activity is most dominantly influenced) by stimulation waveforms with a large low-frequency component (denoted by the striped area on the graph). The −3 dB cutoff frequency Fc for the neural low-pass filter is in the range of 200-500 Hz. The use of bi-phasic stimulation pulses between electrodes positioned outside the brain leads to the target brain area experiencing rapid voltage gradients of opposite polarity (i.e., high-frequency stimulation), which is largely filtered out by the low-pass filtering property of the neuron. Therefore, stimulation waveforms that have rapid changes in voltage gradients can be less effective at stimulating neurons.

FIG. 6A shows a brain stimulation system including electrode arrays 810, 820 coupled to supplemental device 850, applying pseudomonophasic current pulses, according to embodiments. As shown, a first current pulse P1 can be directed from a first primary electrode 812 of a first electrode array 810 (e.g., the anode) to a second primary electrode 822 of a second electrode array 820 (e.g., the cathode) such that a charging current CC flows through target tissue of the brain 802. Then, a second pulse P2 with an opposite polarity can be directed from one or more secondary electrodes 814 of the first electrode array 810 (e.g., now the anode(s)) to the first primary electrode 812 of the first electrode array (e.g., now the cathode) such that a discharge current DC flows through non-target tissue of the brain (e.g., tissue local to the first electrode array 810). The discharge current DC would counteract the stimulating voltage gradient established by the charging current CC if the discharge current DC was directed through the target brain region 802. When the discharge current DC is not directed between the first primary electrode 812 and the second primary electrode 822, the target brain tissue 802 is not affected by the discharge current DC. FIG. 6B is a diagram showing electric field (e.g., the magnitude of the electric field) across the target brain tissue 802 when pseudomonophasic stimulation is applied. Pseudomonophasic stimulation pulses, from the perspective of the brain target, increase the potency of the neuromodulation effect while maintaining charge-balanced stimulation at the electrode site. As shown, the electric field generated at the target tissue 802 during the discharging phases does not counteract the electric field generated at the target tissue 802 during the charging phases. For example, the electric field generated at the target tissue 802 can be maintained at least 0 V/m during the first pulse P1 and the second pulse P2. FIG. 6C is a graph of membrane potential at an axon hillock of a neuron during application of pseudomonophasic current pulses, according to embodiments. Pseudomonophasic pulses, like monophasic pulses, at the brain target accumulate more quickly to reach the threshold of activation for firing of action potentials than biphasic stimulation pulses. For example, a stimulation yield of pseudomonophasic pulses may be about 2 times to about 20 times more potent and/or effective than biphasic pulses (e.g., pseudomonophasic pulses may be 2 times to about 20 times more potent at polarizing the membrane than biphasic stimulation). By using “monophasic” pulses across distant electrodes and “biphasic” pulses locally at the electrode for charge balancing, a 10× increase in stimulation efficacy may occur. In some embodiments, the stimulation efficacy may increase by about 2× to about 20×, inclusive of all ranges and subranges therebetween.

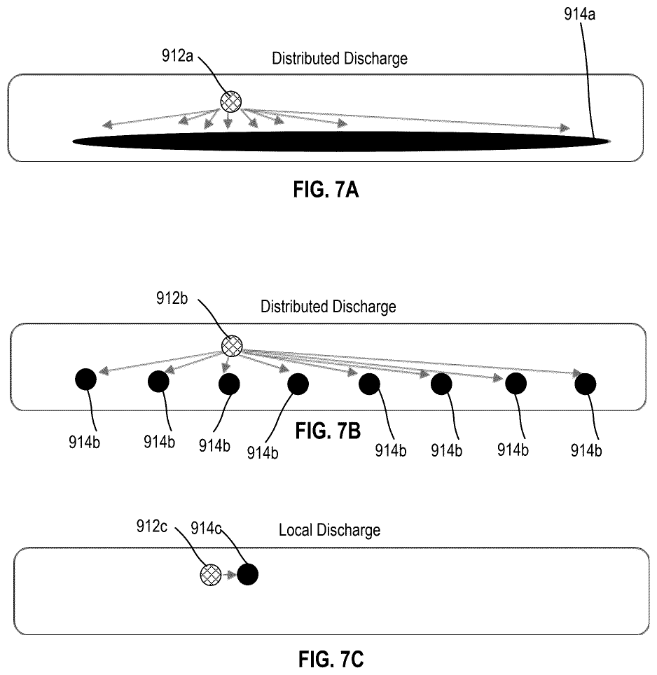

FIG. 7A shows a diagram of distributed current flow when a large discharge partner electrode is used to discharge current. As shown, a stimulation device may include a first electrode 912a and one or more discharge partner electrodes (e.g., a secondary electrode, auxiliary electrode, return electrode, etc.) 914a. A discharge current (e.g., charge of opposite polarity to the charge phase current) may flow between the first electrode 912a and the discharge partner electrode 914a to charge balance the electrode interface of the first electrode 912a. As shown in FIG. 7A, the discharge partner electrode is large, elongated electrode. As described herein, large electrodes refer to electrodes about 20 mm to about 100 mm in diameter. For example, the discharge partner electrode 914a may be a strip electrode or a long, exposed return electrode.

As shown in FIG. 7B, the discharge partner electrodes 914b include a plurality of discrete electrodes spaced along an array. As shown in FIG. 7B, the discharge current flows between a first electrode 912b and a plurality of discharge partner electrodes 914b. In both FIGS. 7A and 7B, the discharge current is distributed across a large area, which is undesirable for stimulation applications. The problem with using discharge partner(s) covering a large area on the strip electrode (e.g., long exposed return electrode or multiple discrete discharge partners connected to a common ground) is that the current flow during discharge is widely distributed in space, leading to poor focality. When the discharge current is widely distributed in space, this can cause undesirable effects on brain tissue such as unwanted stimulation and/or inhibition across a large region of brain tissue. These problems can be avoided by using a local, near-by electrode as the discharge partner, in accordance with embodiments set forth herein.

FIG. 7C shows a diagram of local, compact current flow when a nearby discharge partner electrode 914c is used to discharge current. In some embodiments, an electrode 912c and the discharge partner electrode 914c may be disposed on the same electrode array at a short distance from one another to prevent current flowing to unwanted regions of brain tissue. Discharging current with a nearby discharge partner electrode 914 ensures or increases the likelihood that discharge current flows through a small/reduced region of brain tissue, reducing a number of neurons impacted by the discharge current, which reduces an overall impact on the brain of the discharge current.

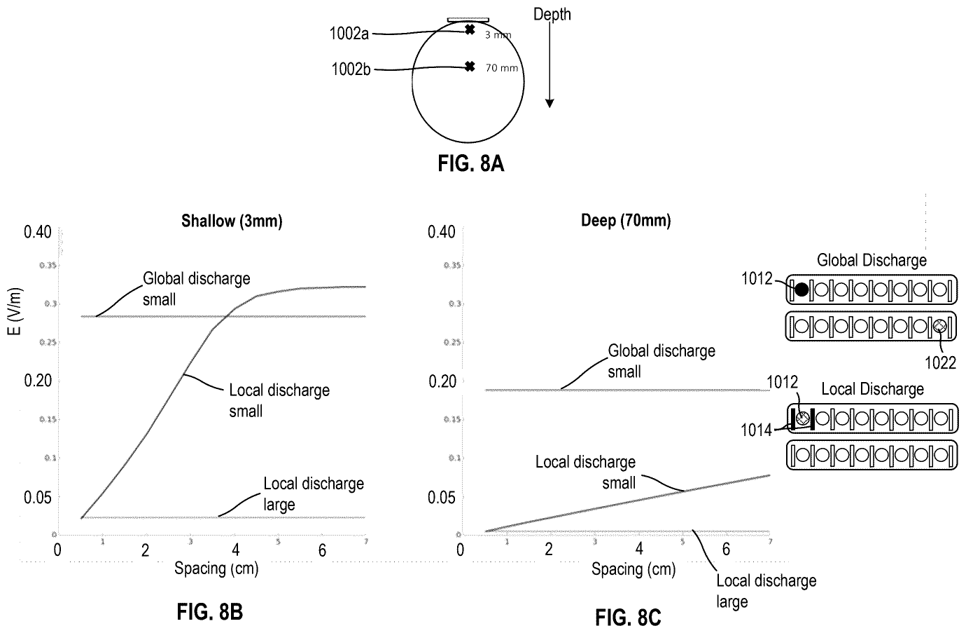

FIG. 8A is a diagram of a shallow stimulation target 1002a at 3 mm under the surface of the brain and a deep stimulation target 1002b at 70 mm under the surface of the brain. FIGS. 8B-8C show the electric field at an applied current of about 1 mA across the target tissue when different spacing between pairs of discharge electrodes are used. The plot illustrates the following discharge strategies: (1) global discharge with a small discharge partner electrode (i.e., “global discharge small”), (2) local discharge with a large discharge partner electrode (i.e., “local discharge large”), and (3) local discharge with a small discharge partner electrode (i.e., “local discharge small”). As used herein, global discharge refers to when a discharge current is caused to flow from an electrode on a first electrode array to an electrode on a second electrode array different than the first electrode array. As used herein, local discharge refers when a discharge current is caused to flow from an electrode on a first electrode array to one or more electrodes on the first electrode array and/or to a return electrode that is nearby the first electrode. In some embodiments, the small discharge partner electrode can refer to an electrode having a diameter in a range of about 0.01 mm to about 2.0 mm, inclusive of all values and subranges therebetween. In some embodiments, the large discharge partner electrode can refer to an electrode having a diameter in a range of about 2 mm to about 15 mm, inclusive of all values and subranges therebetween.

As shown, when the global discharge with a small discharge partner electrode is used, the magnitude of the electric field created by the discharge, which is counteractive to stimulation, is at a constant high level (e.g., above 0.25 volts per meter (V/m)) regardless of the spacing between the electrodes. When the local discharge with a large discharge partner electrode is used, the electric field created by the discharge is at a constant low level (e.g., about 0 V/m to about 0.05 V/m) at an applied current of about 1 mA. This trend occurs because the discharge current is generally more dispersed, and therefore, current density across any one region of the brain is reduced. When the local discharge with a small discharge partner electrode is used, the electric field at the target tissue generated by the discharge current increases with increasing space between the electrodes. The general trends of each discharge approach are similar for both the shallow stimulation target 1002a and the deep stimulation target 1102b. Therefore, for maximal stimulation efficiency at the target brain tissue, the distance between the stimulation electrode and the local discharge partner electrode should be smaller than a distance between the stimulation electrode and the target brain tissue. For example, for a target tissue 3 mm below the surface of the brain, the discharge electrode can be less than about 1 cm from the stimulating electrode. For a target tissue 70 mm below the surface of the brain, the discharge electrode can be less than about 4 cm from the stimulating electrode. In some embodiments, a closer distance between the stimulating electrode and the local discharge partner provides superior stimulation efficiency than further distances. In some embodiments, a distance between the stimulation electrode and the local discharge partner electrode may be in a range of about 0.50 mm to about 5 cm, inclusive of all values and subranges therebetween. In some embodiments, the distance between the stimulation electrode and the local discharge partner may be less than 1 cm.

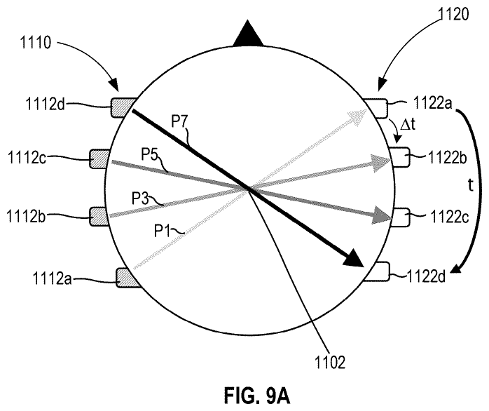

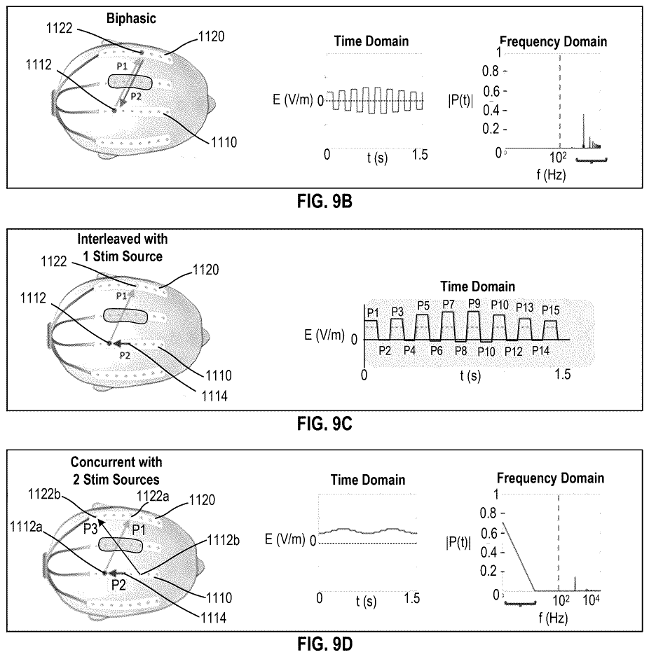

FIG. 9A shows a brain stimulation protocol where stimulation is applied to a target region 1102 by causing current to flow from a first set of primary electrodes 1112a, 1112b, 1112c, 1112d in a first electrode array 1110 to a second set of primary electrodes 1122a, 1122b, 1122c, 1122d in a second electrode array 1120 over a period of time t, with time increments of Δt. As shown, P1, P3, P5, and P7 refer to charging phase current pulses for stimulating the target region 1102. As shown, a pair of electrodes to be activated can be determined based on the relative position of the pair of electrodes to the target brain region such that current is directed through the target brain region 1102. The electrodes 1112a-1112d, 1122a-1122d can be activated according to different stimulation waveforms as described in FIGS. 9B-9D.

FIG. 9B shows the resulting waveform (middle panel) from applying the brain stimulation protocol of FIG. 9A at an applied current of about 1 mA using interleaved (sequential) stimulation with a biphasic stimulation waveform and one current source. Using this method, a charge current is directed from a primary electrode 1112 in the first electrode array 1110 to a primary electrode 1122 in the second electrode array 1120 (e.g., active charging), and a discharge current is directed from the primary electrode 1122 in the second array 1120 to a primary electrode 1112 in the first electrode array 1110 (e.g., active discharging). In the time domain (middle panel), the biphasic pulse pattern causes the electric field (i.e., voltage gradient) across the target region 1102 to oscillate between positive and negative values, meaning that the electric field created during the discharging phases counteracts the electric field created during the charging phase. This positive and negative electric field oscillation results in a high frequency electric field (right panel). The high frequency electric field delivered to the target region 1102 can be above 10² Hertz (Hz), which is above the high-frequency cut-off frequency of a typical neuron (e.g., above the cut-off stimulation frequency at which the neuron can be effectively modulated).

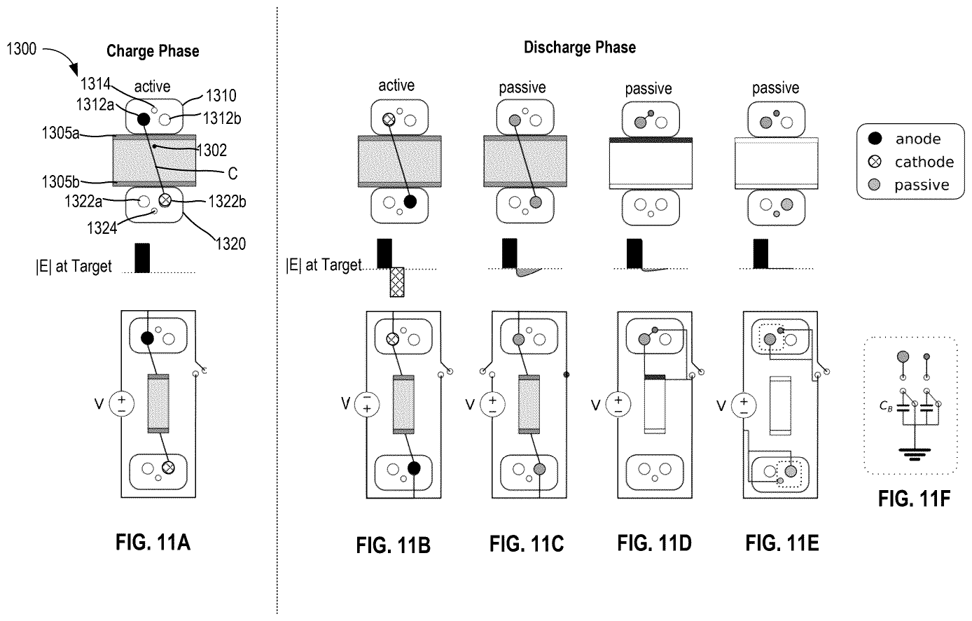

FIG. 9C shows the resulting waveform (right panel) at the target region 1102 from applying the brain stimulation protocol of FIG. 9A at an applied current of about 1 mA using a pseudomonophasic stimulation method with interleaved (sequential) stimulation with one current source. Using this method, a charge current is directed from a primary electrode 1112 in the first electrode array 1110 to a primary electrode 1122 in the second electrode array 1120 (e.g., active charging), and a discharge current is directed from a secondary 1114 electrode in the first electrode array 1110 to the primary electrode 1112 in the first electrode array 1110, as shown in the left panel. In some embodiments, the discharging phase may be local (e.g., pass through non-target tissue that is near the discharging electrodes). The discharge phase of FIG. 9C is shown in further detail in FIG. 11E. In the time domain, the pseudomonophasic stimulation with one current source causes the electric field at the target brain region to oscillate between approximately zero electric field (V/m) and positive peak values. Therefore, the electric field created during the discharging phases does not fully counteract or cancel out the electric field created during the charging phase.

FIG. 9D shows the resulting stimulation waveform (middle panel) from applying the brain stimulation protocol of FIG. 9A at an applied current of about 1 mA using a concurrent (temporally overlapping) pseudomonophasic stimulation method with two current sources. Using this method, a first charge current can be directed from a primary electrode 1112a in the first electrode array 1110 to a primary electrode 1122a in the second electrode array 1120, and a first discharge current is directed from a secondary 1114 electrode in the first electrode array 1110 to the primary electrode 1112a in the first electrode array 1110, as shown in the left panel. At the same time (concurrently) that the first discharge current is directed from the secondary 1114 electrode in the first electrode array 1110 to the primary electrode 1112a in the first electrode array 1110, a second charge current can be directed from a second primary electrode 1112b in the first electrode array 1110 to a second primary electrode 1122b in the second electrode array 1120. Subsequently, a second discharge current (not shown in FIG. 9D) can be used to return the electrical charge to the primary electrode 1112b in the first electrode array 1110. In the time domain (middle panel), the concurrent pseudomonophasic stimulation causes the electric field across the target region to remain positive and have much smaller fluctuation in magnitude than the biphasic stimulation and pseudomonophasic stimulation with one current source. As shown, the electric field remains substantially positive (above zero) and changes gradually with small increments. Therefore, the electric field at the target region 1102 remains within a narrower range (all positive in polarity) over time during the stimulation protocol, thereby improving effectiveness in exciting neurons in the target region 1102. In some embodiments, the electric field at the target region 1102 can be maintained within a predefined range throughout an entirety of the stimulation waveform. In the frequency domain (right panel), the stimulation waveform created by using pseudomonophasic stimulation with two current sources is below the low-frequency cut-off threshold for neurons (i.e. is within the pass-band of the neural filter); and therefore, concurrent pseudomonophasic stimulation has a higher likelihood of modulating the activity of the neurons in the target region 1102.

FIG. 10A shows a system for providing brain stimulation including a first electrode array 1210 including a first primary electrode 1212a, a second primary electrode 1212b, and a secondary electrode 1214. The system 1200 further includes a second electrode array 1220 including a first primary electrode 1222a, a second primary electrode 1222b, and a secondary electrode 1224. In some embodiments, the first electrode array 1210 is disposed on a first side of target brain tissue and the second electrode array 1220 is disposed on a second side of target brain tissue 1202. FIG. 10B on the left shows a diagram of global stimulation between the first primary electrode 1212a of the first electrode array 1210 and the second primary electrode 1222b of the second electrode array 1220. FIG. 10B on the right shows local stimulation between the first primary electrode 1212a of the first electrode array 1210 and the secondary electrode 1214 of the first electrode array 1210. As shown, during global stimulation, current flows through the target region of the brain 1202. During the local stimulation shown, current flows through the local tissue 1205a near the first electrode array 1210.

FIG. 10C shows examples of charging and discharging current pulses. As shown, the charging phase occurs during the shaded current pulses and the discharging phase occurs during the non-shaded current pulses. The charging phases have positive current, and the discharging phases have negative current. In some embodiments, the discharging current pulses can have an equal and opposite magnitude and shape as the charging current pulses, as shown in FIG. 10C panel (a). In some embodiments, the discharging current pulses can have a lower magnitude over a longer period of time such that the integral over time of the discharging pulse(s) (i.e., the total area under the curve, which is the total electrical charge) equals the integral over time of the charging pulse(s), as shown in FIG. 10C panel (b). The current during the discharging phase can be directed through the electrode interface and to one or more discharge partners (e.g., secondary electrodes and/or ground electrodes). In some embodiments, the discharging current may be passive and gradually decrease over time, as shown in FIG. 10C panel (c). In some embodiments, a plurality of charging pulses may be used having different magnitudes, and the discharging current pulse may be configured to charge balance the plurality of charging pulses, as shown in FIG. 10C panel (d).

FIG. 11A shows a brain stimulation system 1300 during a charging phase (top) and a circuit diagram of the brain stimulation device 3100 in the charging phase (bottom). As shown, the brain stimulation device 1300 includes a first electrode array 1310 with a first primary electrode 1312a, a second primary electrode 1312b, and a secondary electrode 1314. The brain stimulation system 1300 further includes a second electrode array 1320 with a first primary electrode 1322a, a second primary electrode 1322b, and a secondary electrode 1324. The first electrode array 1310 may be disposed adjacent to target brain tissue 1302 on a first side and the second electrode array 1320 may be disposed adjacent to target tissue 1302 on a second side. The brain stimulation system 1300 may be functionally and/or structurally similar to any brain stimulation system described herein, and therefore, certain aspects of the brain stimulation system are not described herein with respect to FIGS. 11A-11F.

In some embodiments, the first and second electrode arrays 1310, 1320 may be paddle electrode arrays used to pass current (black line C) across the through the skull and across the brain. In some embodiments, the primary electrodes 1312a-1312b, 1322a, 1322b may be larger in size than the secondary electrodes 1314, 1324. The dark shading represents positive current flow into the brain at the anode and the cross-hatching represents negative current flow out of the brain at the cathode (as shown in the legend). The electric field magnitude |E| generated at the target 1302 is shown for the charging phase (middle). FIG. 11A on the bottom shows an equivalent circuit of the brain stimulation system where current flows between the anode and the cathode when a voltage is applied across the tissue such that |E| is generated at the target tissue in the brain as described above.

Dring the charging phase, system 1300 is configured to apply global stimulation. For example, current can flow from the first primary electrode 1312a of the first electrode array 1310 (e.g., the anode) through the target region of the brain 1302 (and the local brain tissue regions 1305a, 1305b), and to the second primary electrode 1322b of the second electrode array 1320 (e.g., the cathode). Although FIG. 11A shows the first primary electrode 1312a of the first electrode array 1310 as the anode, and the second primary electrode 1322b of the second electrode array 1320 as the cathode, any of the primary electrodes can be configured as the anode or the cathode.

FIGS. 11B-11E show different discharging techniques for the brain stimulation system 1300 (top) and circuit diagrams of the discharging techniques (bottom). FIG. 11B (bottom) shows global active discharge of the anode by flipping the polarity of the voltage source such that current flows from the cathode to the anode. However, this method allows charge of opposite polarity to flow through the target brain tissue, and therefore an electric field (with a magnitude of |E|) would be applied to the target brain tissue that counteracts the stimulating electric field magnitude (middle). FIG. 11C (top) shows an example of global passive discharge with the electrodes that are passively discharging shown in grey (light shading). As shown in FIG. 11C (bottom), the voltage source can be disconnected from the brain tissue, and the first primary electrode 1312a of the first electrode array 1310 and the second primary electrode 1322b of the second electrode array 1320 can be shorted to ground. Global passive discharge causes charge with opposite polarity to slowly flow through the target brain 1302 tissue over a longer amount of time, shown in FIG. 11C (middle). FIG. 11D (top) shows local passive discharge between one primary electrode 1312a and the secondary electrode 1314 on the same electrode array 1310 by shorting these electrodes to ground. This directs most of the current through the local tissue 1305a adjacent to the first electrode array 1310 rather than the target brain tissue 1302. FIGS. 11E-11F shows internal passive discharge in which discharge only occurs internally. This is done by disconnecting the electrode wires from the tissue and connecting/shorting all the inline block capacitors (CB) to ground, as shown in FIG. 11F. Block capacitors are included in series with each electrode to prevent direct current (DC) that can cause irreversible and damaging electrochemical reactions.

FIG. 12A shows a method of providing interleaved brain stimulation using a brain stimulation system 1400 with one current source, according to embodiments. Each electrode array 1410, 1420 has two larger primary electrodes 1412a, 1412b, 1422a, 1422b and one smaller secondary electrode 1414, 1424. Tissue nearby the electrode 1405a, 1405b is denoted as local tissue. Dark shading represents positive current flow into the brain at the anode, cross-hatching represents negative current flow out of the brain at the cathode, and grey indicates passive current flow (as shown in the legend). Current flows between anode and cathode when a voltage is applied across the tissue during a charging phase (bottom), and an electric field (with a magnitude of |E|) is generated at a nearby target in the brain (middle). Each electrode pair stimulates globally across the tissue and then discharges passively. For example, after the charging phase, discharging phase occurs in which anode and cathode are disconnected from brain tissue and internal passive discharging occurs. The electric field magnitude is non-zero during active stimulation and zero during internal passive discharge because the primary electrodes are disconnected from the tissue, so no current flows though the target brain tissue.

FIG. 12B shows a method for providing concurrent brain stimulation using the brain stimulation system 1400 with one current source, according to embodiments. The method of FIG. 12B is similar to that of FIG. 12A except charging and discharging phases can be concurrent. For example, when one pair of primary electrodes are active, the other pair of primary electrodes and secondary electrodes are passively discharging. In some embodiments, when concurrent stimulation is being performed with one current source, the passive discharge is internal (see FIG. 11F) so that the drain lines to ground do not draw current from the primary active electrodes. As shown in FIG. 12B (middle), the electric field magnitude generated at the target brain tissue through both the charging phases and the discharging phases.

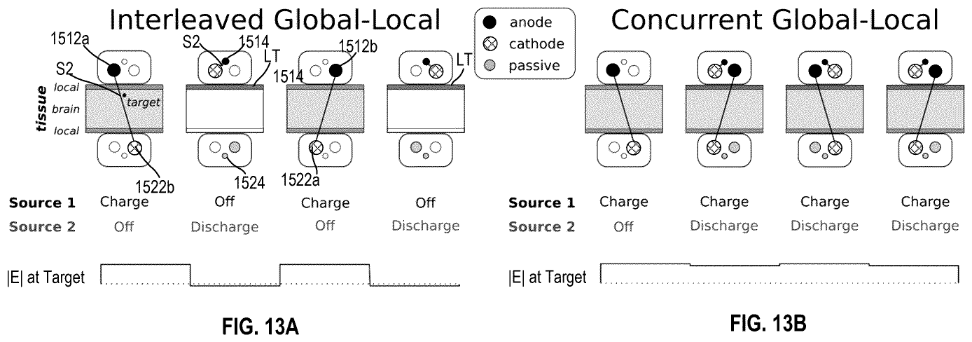

FIG. 13A shows a method for providing interleaved brain stimulation using a brain stimulation device with two current sources S1, S2, according to embodiments. Each electrode array has two larger primary electrodes and one smaller secondary electrode. Tissue nearby the electrode is denoted as local tissue. Dark shading represents positive current flow into the brain at the anode, cross-hatching represents negative current flow out of the brain at the cathode, and grey indicates passive current flow. Current flows between the anode and cathode when a voltage is applied across the tissue.

As shown, current (black line C) is passed globally across the tissue between a first pair of primary electrodes (e.g., across electrode arrays) 1512a, 1522b. The top primary electrode 1512a discharges actively to its nearby secondary electrode 1514, while concurrently, the bottom primary electrode 1522b discharges internally and passively. Local and brain tissues targeted by Source 1 are shown in shades of grey. The local tissue targeted by Source 2 is shown labeled LT. This sequence is repeated with the second pair of primary electrodes across the electrode arrays 1512b, 1522a. An electric field (with a magnitude of |E|) is generated at a nearby target in the brain (bottom)

FIG. 13B shows a method for providing concurrent brain stimulation using two current sources, according to embodiments. The method of FIG. 13B is similar to that of FIG. 13A except that charging and discharging occur concurrently. When one pair of primary electrodes is active, the other pair of primary electrodes and secondary electrodes are discharging. Only two sources can be used simultaneously, therefore, the bottom electrode array discharges passively.

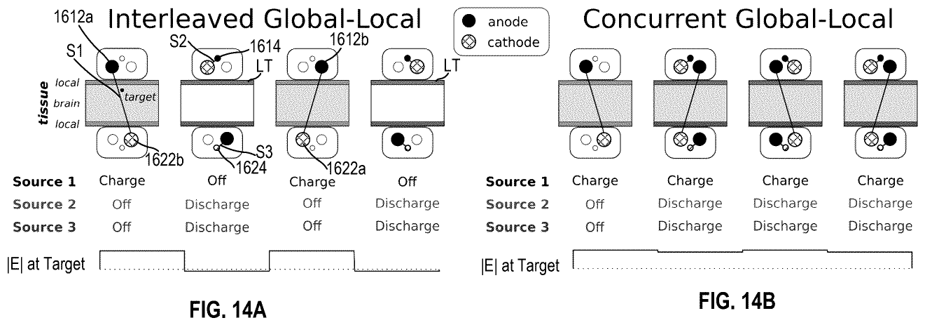

FIG. 14A shows a method for providing interleaved brain stimulation using a brain stimulation device with three current sources, according to embodiments. Dark shading represents positive current flow into the brain at the anode, cross-hatching represents negative current flow out of the brain at the cathode, and grey indicates passive current flow. As shown, Source 1 S1 causes active global stimulation between a pair of primary electrodes 1612a, 1622b across electrode arrays. An electric field (with a magnitude of |E|) is generated at a nearby target in the brain (bottom). Sources 2 S2 and Source 3 S3 cause local active discharge of the top and bottom electrodes arrays, respectively. As shown, during discharge phases, the top primary electrode 1612a discharges to its nearby secondary electrode 1614 while the bottom primary electrode 1622b discharges simultaneously to its nearby secondary electrode 1625. Then the charging phase and discharging phase are repeated with a second pair of primary electrodes 1612b, 1622a.

FIG. 14B shows a method for providing concurrent brain stimulation using three current sources, according to embodiments. The method of FIG. 14B is similar to that of FIG. 14A except that charging and discharging is concurrent. While one primary electrode pair passes current globally through the brain, the other primary electrodes locally and actively discharge to their respective secondary electrode. Two independent sources are needed to discharge to primary-secondary pairs to keep the two separate current paths local.

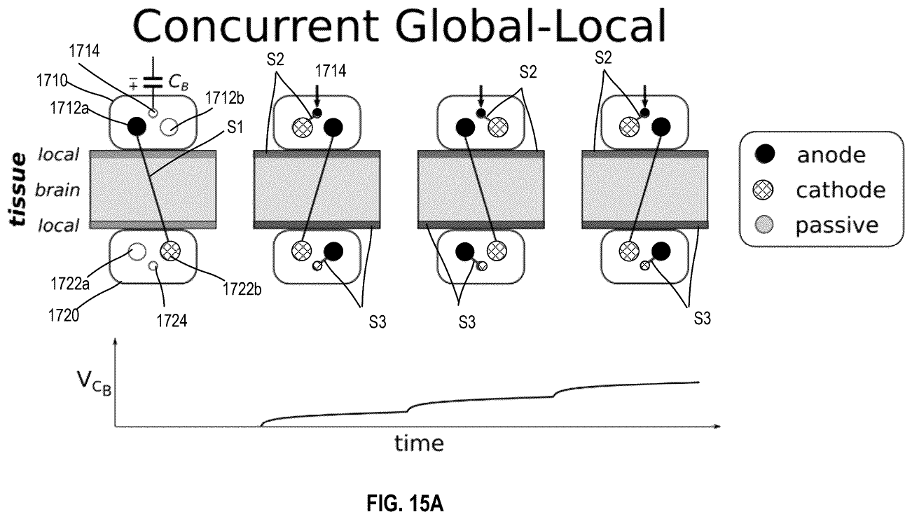

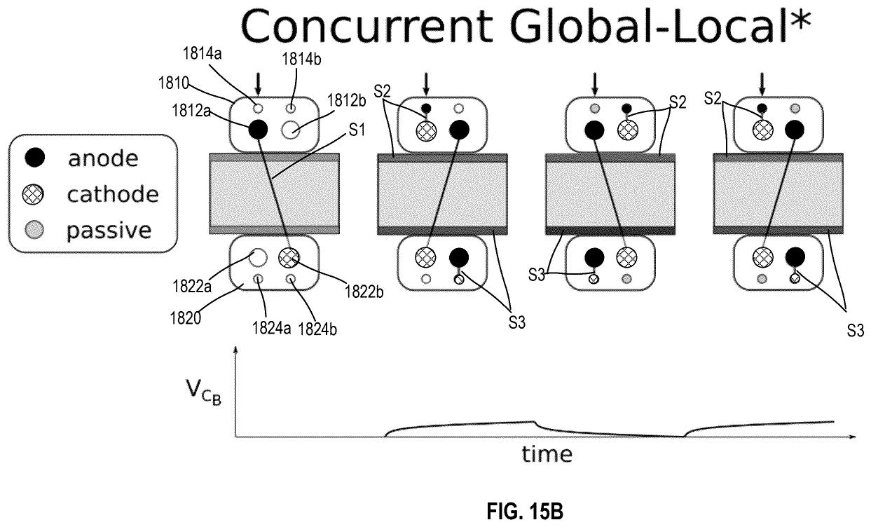

FIGS. 15A-15B show a method for discharging secondary electrodes in a brain stimulation system, according to embodiments. Each electrode array 1710, 1720 has two larger primary electrodes 1712a, 1712b, 1722a, 1722b and one smaller secondary electrode 1714, 1724. The voltage time series (bottom) is of a representative secondary electrode 1714 in the top electrode array 1710. Dark shading represents positive current flow into the brain at the anode, cross-hatching represents negative current flow out of the brain at the cathode, and grey indicates passive current flow (current inward or outward across a short to ground). The current path of one current source is shown in black S1 and grey. The current paths of the second and third sources are labeled S2 and S3, respectively. As shown in FIG. 15A, charge builds up on the blocking capacitor (C_{B}) in line with the secondary electrode 1714 if it is only a dedicated source or sink. A voltage builds up across the blocking capacitor as it charges (V_{CB} bottom). As shown in FIG. 15B, each electrode array 1810, 1820 has two larger primary electrodes 1812a, 1812b, 1822a, 1822b and two smaller secondary electrode 1814a, 1814b, 1824a, 1824b. Charge build up is resolved by having one smaller secondary electrode for every larger primary electrode. The charge and voltage buildup on the secondary electrodes can be passively discharged internally when not in use (bottom).

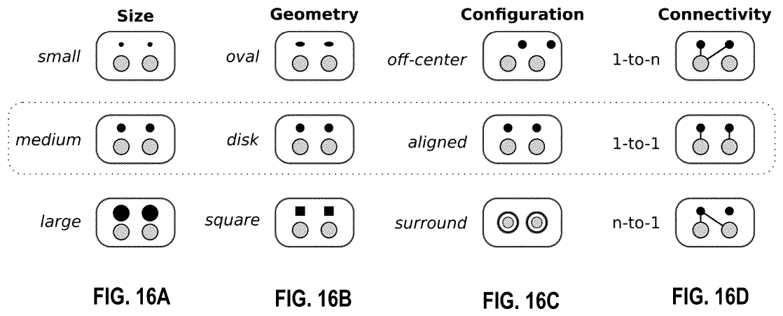

FIGS. 16A-16D shows different designs for secondary or auxiliary electrodes of a brain stimulation device, according to embodiments. As shown in FIG. 16A, the auxiliary electrode (filled black circles) can be small (top), medium (middle), or large (bottom) compared to the primary electrodes (filled grey circles). For example, the auxiliary electrodes can have a small size such that they are about 25% less than the diameter or area of the primary electrodes. In some embodiments, the auxiliary electrodes can have a medium size such that they are between about 25% to about 75% the diameter or area of the primary electrodes. In some embodiments, the auxiliary electrodes can have a large size such that they are substantially equivalent in size to the primary electrodes. The size of the auxiliary electrode(s) can be adjusted so that the impedance between two primary electrodes matches the impedance between primary and auxiliary electrodes. In some embodiments, the auxiliary electrodes across arrays may have the same size. In some embodiments, the auxiliary electrodes across arrays may have different sizes.

As shown in FIG. 16B, the auxiliary electrodes can have any suitable shape such as, for example ellipsoidal (top), circular or disk-like (middle), or rectangular (bottom). As shown in FIG. 16C, auxiliary electrodes can be placed off-center in between the primary electrodes (top), aligned with the center of each primary electrode (middle), or surrounding the primary electrode with an annular geometry (bottom). In some embodiments, the auxiliary electrode can be disposed between primary electrodes in an array. As shown in FIG. 16D, current can pass between one primary electrode connected to many auxiliary electrodes (top), one primary electrode connected to one auxiliary electrode (medium), and many primary electrodes connected to one auxiliary electrode (bottom). The design choices in the middle row are represented in the other figures.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified, and such modification are in accordance with the variations of the invention. Additionally, certain of the steps may be performed concurrently in a parallel process, when possible, as well as performed sequentially as described above. The embodiments have been particularly shown and described, but it will be understood that various changes in form and details may be made.

1. A method for electrically stimulating target tissue of a brain using at least a first array of electrodes and a second array of electrodes different from the first array of electrodes, the first array of electrodes including a first primary electrode and one or more secondary electrodes, the second array of electrodes including a second primary electrode and one or more secondary electrodes, the method comprising:
causing, during a charging phase, a current to flow between the first primary electrode and the second primary electrode, each of the first array of electrodes and the second array of electrodes disposed to at least one of a side of, beside, or close in proximity to a target tissue of the brain such that current passes through the target tissue of the brain; and
causing, during a discharging phase, a majority of a discharge current to pass through non-target tissue of the brain, the discharge current flowing between at least one of:
(i) two or more electrodes within the first array of electrodes,
(ii) two or more electrodes within the second array of electrodes,
(iii) at least one electrode from the first array of electrodes and a return electrode separate from the first array of electrodes,
(iv) at least one electrode from the second array of electrodes and a return electrode separate from the second array of electrodes.

2. The method of claim 1, wherein the causing the current to flow between the first primary electrode and the second primary electrode results in a first charge at a first electrode interface, and the causing the at least one of (i), (ii), (iii), or (iv) generates a second charge at the first electrode interface equal in magnitude and opposite in polarity to the first charge at the first electrode interface.

3. The method of claim 1, wherein the method further comprises:
repeating at least one of the charging phases or discharging phases in a predefined sequence, thereby defining a plurality of charging phases and a plurality of discharging phases.

4. The method of claim 3, wherein during each charging phase an associated current thereof has a first polarity, and during each discharging phase an associated current thereof has a second polarity opposite to the first polarity.

5. The method of claim 4, wherein at least one of:
no charging phase from the plurality of charging phases overlaps in time with any other charging phase from the plurality of charging phases; or
no charging phase from the plurality of charging phases overlaps in time with any discharging phase from the plurality of discharging phases.

6. The method of claim 4, wherein at least one of:
at least one charging phase from the plurality of charging phases overlaps in time with at least one other charging phase from the plurality of charging phases; or
at least one charging phase from the plurality of charging phases overlaps in time with at least one other discharging phase from the plurality of discharging phases.

7. The method of claim 4, wherein during each discharging phase from the plurality of discharging phases, the discharge current is a passive discharge current in which the discharge current flows between at least one of (iii), or (iv)

8. The method of claim 4, wherein during each discharging phase from the plurality of discharging phases, the discharge current is an active discharge current in which the discharge current flows between at least one of (i) or (ii).

9. The method of claim 1, wherein a maximum duration of the charging phase is less than or equal to 500 milliseconds (ms).

10. The method of claim 1, wherein a within-array distance is a distance between edges of any two electrodes within a respective array of electrodes, a shortest within-array distance of the first array of electrodes is less than 1 centimeter (cm) and a shortest within-array distance of the second array of electrodes is less than 1 cm.

11. The method of claim 1, wherein an across-array distance is a shortest distance between edges of any electrode in the first array of electrodes and edges of any electrode in the second array of electrodes, a ratio of the across-array distance to a within-array distance is greater than or equal to 2.

12. The method of claim 1, wherein the discharge current flowing between at least one of (i), (ii), (iii), or (iv)prevents at least one electrochemical reaction from occurring in brain tissue near at least one of the first primary electrode or the second primary electrode.

13. The method of claim 4, wherein the repeating at least one of the charging phase or the discharging phase in a predefined sequence generates at least a portion of a waveform for modulating the target tissue of the brain, the waveform having a frequency configured to modulate neural activity.

14. The method of claim 13, wherein the frequency is less than 250 Hz.

15. The method of claim 1, wherein during the discharge phase, the discharge current flows between at least one electrode from the first array of electrodes and a return electrode through a wire.

16. The method of claim 1, wherein during the discharge phase, the discharge current flows between at least one electrode from the second array of electrodes and a return electrode through a wire.

17. A method for electrically stimulating target tissue of a brain using at least a first array of electrodes and a second array of electrodes different than the first array of electrodes, the first array of electrodes including a first primary electrode, the second array of electrodes including a second primary electrode, the method comprising:
causing, during a charging phase, current to flow between the first primary electrode and the second primary electrode, each of the first array of electrodes and the second array of electrodes disposed to one side of a target tissue of the brain such that current passes through the target tissue of the brain; and
causing, during a discharging phase, a majority of the discharge current to pass through non-target tissue of the brain, the discharge current flowing between at least one of:
(i) the first primary electrode of the first electrode array and a return electrode separate from the first array of electrodes, or
(ii) the second primary electrode of the first electrode array and a return electrode separate from the second array of electrodes.
